(19) United States
(12) Patent Application Publication
(10) Pub. No.: US 2003/0180723 A1
(43) Pub. Date: Sep. 25, 2003

(54) Cloning of novel gene sequences expressed and repressed during winter dormancy in the apical buds of tea ( Camellia sinensis L. (O.) Kuntze) bush

(76) Inventors: Sanjay Kumar, Palampur (IN); Lakhvir Lal, Palampur (IN); Paramvir Singh Ahuja, Palampur (IN)

Correspondence Address: Allan Ratner, RatnerPrestia, PO Box 980, Valley Forge, PA, 19482-0980, US

(21) Appl. No.: 09/823,887

(22) Filed: Mar. 31, 2001

(51) Int. Cl. C12Q 1/68; C07H 21/04; C12P 21/02; C12N 5/04

(52) U.S. Cl. 435/6; 435/69.1; 435/320.1; 435/419; 536/23.6; 530/350

(57) ABSTRACT

The present invention relates to cloning of novel gene sequences expressed and repressed during winter dormancy in the apical buds of Camellia sinensis L. (O.) Kuntze (hereinafter, referred to tea) bush, particularly, relates to identification, cloning and analysis of novel 3 prime (hereinafter called as 3′) ends of the genes (gene in the present invention refers to the deoxyribonucleic acid (hereinafter known as, DNA) sequences that are expressed and repressed in winter-dormant apical buds of tea. 3′ end refers to that end of DNA which has free hydroxyl group at 3rd position of the carbohydrate moiety of the DNA molecule.

### CROSS-REFERENCE TO RELATED APPLICATIONS

Not applicable.

### STATEMENTS REGARDING FEDERALLY SPONSORED RESEARCH OR DEVELOPMENT

Not applicable.

### REFERENCE TO A MICROFICHE APPENDIX

Not applicable.

### BACKGROUND OF THE INVENTION

1. Field of Invention

The present invention relates to cloning of novel gene sequences expressed and repressed during winter dormancy in the apical buds of Camellia sinensis L. (O.) Kuntze (hereinafter, referred to tea) bush. Particularly, this invention relates to identification, cloning and analysis of novel 3 prime (hereinafter called as 3′) ends of the genes (gene in the present invention refers to the deoxyribonucleic acid (hereinafter known as, DNA) sequences that are expressed and repressed in winter-dormant apical buds of tea. 3′ end refers to that end of DNA which has free hydroxyl group at 3rd position of the carbohydrate moiety of the DNA molecule.

2. Background and Prior Art References to the Invention

Tea (Camellia sinensis L. (O.) Kuntze) is a perennial plant grown in 31 countries located between 45° N-30° S and 150° E-60° W. Being perennial, the plant experiences several cycles of summer, winter and rainy seasons. Apical bud and the associated two leaves (hereinafter, referred to BATS) are used for tea manufacture. BATS are harvested at frequent interval, depending upon their availability. Prevailing environmental conditions (such as temperature, sun-shine hours and soil and atmospheric moisture etc.) and tea clone determine the availability of BATS for the purpose of harvesting.

A temperature of 30° C. and a day length of 13 hours are considered ideal for growth of the plant. As the temperature drops below 13° C. and day length becomes shorter than 11 hours and 15 minute, the growth and development of BATS is stopped. This phenomenon of suspension of the growth of BATS is termed as winter dormancy. Unlike deciduous plants, tea does not shed any leaf and remains green during dormancy period (Barua, D. N. 1989. Science and practice in tea culture. Tea Research Association, Calcutta, India. 509 p.). Winter dormancy is a universal phenomenon occurring in tea crops grown under the environmental conditions described above (Barua, D. N. 1989. Science and practice in tea culture. Tea Research Association, Calcutta, India. 509 p.). The following Chart 1 illustrates the period of winter dormancy in various tea growing countries across the globe:

CHART 1

| | Dormancy | | | |
|---|---|---|---|---|
| Country/Place | Period | | Location | |
| Georgia, U.S.S.R. | 6 | months | 42° | N |
| Turkey | 6 | months | 41° | N |
| Iran | 5-6 | months | 37° | N |
| North-East India | 3-4 | months | 26° | N |
| Mauritius | 2-3 | months | 20° | S |
| Argentina | 3-4 | months | 30° | S |
| Himachal Pradesh | 5-6 | months | 37° | N |

(Data adapted from Barua, D. N. 1989. Science and practice in tea culture. Tea Research Association, Calcutta, India. 509 p.; Dudeja, V. 1992. Tea Today 13: 1-5).

Temporary suspension of growth or the dormancy is not only limited to apical buds as in the case of tea but, is prevalent in other plats and systems as follows:

a) apical buds of Taxus species (Janes; Harry, W.; Gore; Gerald, E.; Wittman; Wayne, K.; Romen; Harry, T. Jul. 1, 1997; U.S. Pat. No. 5,642,587), beach (Fagus sylvatica; Heide, O. M. 1993. Physiol. Plant. 89: 187-191); grapevine (Vitus vinifera; Nir, G., Shulman, Y., Fanberstein, L. and Lavee, S. 1986. Plant Physiol. 81: 1140-1142; Or, E., Vilozny, I., Eyal, Y. and Ogrodovitch, A. 2000. Plant Mol. Biol.43: 483-494); peach, (Marquat, C., Vandamme, M., gendraud, M. and Petel, G. 1999. Sci. Hort. 79: 151-162; Faye, F. and Floc'h, F. L. J. 1999. Plant Physiol. 154: 471-476); rose (Rosa hybrida; Bris, M. L., Michaux-Ferrière, N., Jacob, Y., Poupet, A., Barthe, P., Guigonis, J-M. and Page-Degivry, M-T. L. 1999. Aust. J. Plant Physiol. 26: 273-281); populus, (Rohde, A., Montagu, M. V. and Boerjan, W. 1996(In Somatic Cell Genetics and Molecular Genetics of Trees. eds Ahuja, M. R., Boerjan, W. and Neale, D. B. Kluwer Academic Publishers. Netherlands. pp 183-188); Jian, L-c., Li, P. H., Sun, L-h and Chen, T. H. H. 1997. J. Exp. Bot. 48: 1195-1207).

b) seeds of douglas fir (Pseudotsuga menziesii; Taylor, M. A., Davies, H. V., Smith, S. B. Abruzzese, A. and Gosling, P. G. 1993. J. Plant Physiol. 142: 120-123; Jarvis, S. B., Taylor, M. A., Bainco, J., Corbineau, F. and Davies, H. V. J. 1997. Plant Physiol. 151: 457-464); barley (Hordeum vulgare; Aalen, R. B. Opsahl-Ferstad, H-G., Linnestad, C. and Olsen, O-A. 1994. Plant J. 5: 385-396; Stacy, R. A. P., Munthe, E., Steinum, T., Sharma, B. and Aalen, R. B. 1996. Plant Mol. Biol. 31: 1205-1216); wild oat (Avena fatua; Li, B. and Foley, M., E. 1995. Plant Mol. Biol. 29: 823-831); tomato, (Groot, S. P. C., and Karssen, C. M. 1992. Plant Physiol. 99: 952-958); cheat (Bromus secalinus; Goldmark, P. J., Curry, J., Morris, C., F. and Walker-Simmons, M. K. 1992. Plant Mol. Biol. 19: 433-441); arabidopsis (Arabidopsis thaliana; Leon-Kloosterziel, K. M., Gil, M. A., Ruijs, G. J., Jacobsen, S. E., Olszewski, N. E., Schwartz, S. H., Zeevaart, J. A. D. and Koornneef, M. 1996. Plant J. 10: 655-661; Haslekas, C., Stacy, R. A. P., Nygaard, V., Culiáñez-Macià and Aalen 1998. Plant Mol. Biol. 36: 833-845); wheat (Triticum aestivum; Morris, C. F., Anderberg, R., J., Goldmark, P. J. and Walker-Simmons, M. K. 1991. Plant Mol. Biol. 1995: 814-821);

c) tubers of potato (Solanum tuberosum; Macdonald, M. M. and Osborne, D. J. 1988. Physiol. Plant. 73: 392-400)

d) corms of gradiolus (Gladiolus grndiflorus; Ginzberg, C. 1973. J. Exp. Bot. 24: 558-566), saffron crocus (Crocus sativus L.; Chrungoo, N. K. and Farooq, S. 1988. Acta Physiol. Plant. 10: 247-255)

e) Bulbs of onion (Abdel-Rahman, M. and Isenberg, F. M. R. 1974. J. agric. Sci. camb. 82: 113-116); tuberose (Polyanthus tuberosa L.; Nagar, P. K. 1995. Scientia Horticulturae. 63: 77-82); tulip (Reea, A R. 1981. Ann. Appl. Biol. 98: 544-548); yam (Dioscorea spp; Hasegawa, K. and Hashimoto, T. 1973. Plant Cell Physiol. 14: 369-377)

Thus, dormancy is exhibited by plant part(s) having meristematic tissue under unfavorable conditions, whether internal or external.

Modulation of dormancy has been and will continue to be a key issue in agriculture system. For example, if seeds or tubers or corms have reduced dormancy periods, before-time germination would lead to a loss to the growers if, the intention was to use them as sowing material. If intended for human or animal consumption, there will be a loss in the nutritional and or processing quality. In either of the cases, economic benefits will not be realized. On the contrary to this, an extended period of dormancy will lead to late germination or sprouting in the grower's field that would affect the crop performance and the crop yield.

Also, one of the major requirements of the tea industry is to have a tea plant with no or at least a reduced period of dormancy in the areas where this problem is prevalent. Nakayama, A. and Harada, S. (Bulletin of the Tea Research Station, Japan, 1962. 1: 28-39) and Barua, 1969 (Two and A Bud.16: 41-45) pointed out that low temperature coupled with short day length were responsible for inducing winter dormancy in tea. Hence, application of gibberellic acid, a plant growth regulator, was recommended to break dormancy. But the action of gibberellic acid was not persistent. Moreover, the results varied with the clones (Das, S. 1977. Ph.D. Thesis, Gauhati University, Assam; Rustogi, P. N. 1980. Two and A Bud. 21: 33). Studies in seeds, tubers and corms also resulted the similar conclusions (Aalen, R. B., Opsahl-Ferstad, H-G., Linnestad, C. and Olsen, O-A. 1994. Plant J. 5: 385-396; Bagni, N., Malucelli, B. and Torrigiana, P. 1980. Physiol. plant. 49: 341-345; DeBottini, G. A., Bottini, R. and Tizio, R. 1982. Oyton. 42: 115-121; Bewley, J. D. and Black, M. 1985. Physiology of development and germination. Plenum Press, New York. pp190-192; Delvallee, I., Paffen, A., and de Klerk, G. J. 1990. Physiol. Plant. 80: 431-436; Djilinov, D., Gerrits, M., Ivanova, A., Van Onikelen, H. A. and Dc Klerk, G. J. 1994. Physiol. Plant. 91: 639-644; Ginzberg, C. 1973. J. Exp. Bot. 24: 558-566; Kurashi, S., Daisuki, Y., Naoki, S. and Shigeki, H. 1998. J. Plant Growth Regul. 8: 3-10).

The above results dictated the adoption of molecular approach. A few dormancy related cDNAs have been cloned from seeds of Bromus and Hordeum vulgare using differential screening (Goldmark, P. J., Curry, J., Morris, C. F. and Walker-Simmons, M. K. 1992. Plant Mol. Biol. 19: 433-441; Aalen, R. B., Opsahl-Ferstad, H-G., Linnestad, C. and Olsen, O-A. 1994. Plant J. 5: 385-396.). Clone pBS128 was isolated from dormant Bromus seeds and the application of ABA to non-dormant seeds resulted in enhanced expression of this transcript (Goldmark, P. J., Curry, J., Morris, C. F. and Walker-Simmons, M. K. 1992. Plant Mol. Biol. 19: 433-441). This pBS128 from Bromus secalinus, which is maintained at a high level in dormant, but not in non-dormant hydrated seeds, has been suggested to play a role in the maintenance of embryo dormancy. Sequence analysis revealed that the encoded protein belongs to recently discovered group of antioxidants called peroxiredoxin (Li, B. and Forey, M. E. 1997. Trends in Plant Sci. 2: 384-389). The sequence analysis did not show any homology to the other reported genes. Interestingly, a transcript B15C present in the embryo of developing barley seeds showed 95% homology with the clone pBS128 (Aalen, R. B., Opsahl-Ferstad, H-G., Linnestad, C. and Olsen, O-A. 1994. Plant J. 5: 385-396.).

Differentially expressed partial cDNA fragments have been cloned from dormant and non-dormant wild oat embryos (Johnson, R. R., Cranston, H. J., Chaverra, M. E. and Dyer, W. E. 1995. Plant Mol. Biol. 28: 113-122). Out of several such clones, two clones namely AFD4 and AFN5 were found to be very interesting. The protein coded by AFD4 (protein Z analogue) was presumed to be a repressor of germination that maintains the seeds in dormant state. AFN5, which was expressed very early during imbibition, might code for a protein to be needed to signal or initiate early steps in germination (Johnson, R. R., Cranston, H. J., Chaverra. M. E. and Dyer, W. E. 1996. Plant dormancy, Physiology, Biochemistry and Molecular Biology. CAB International, Wallingford, UK. pp. 293-300). Other embryo specific genes that are induced by the application of ABA have also been identified (Hatzopoulos, P., Fong, F. and Sung, Z. R. 1990. Plant Physiol. 94: 690-695; Vance, V. B. and Huang, A. H. C. 1988. J. Biol. Chem. 263: 1476-1481), but their correlation with dormancy was not established.

The following Chart 2 illustrates the status of information available on the phenomenon of dormancy in plants:

CHART 2

| Character, | | | |
|---|---|---|---|
| Property, Trait, | | | |
| Enzyme, Gene | Criteria/Objective | System adopted | Reference |
| β-Amylase and | Starch catabolism | Resting fronds | Luka, Z. A., |
| starch | affecting dormancy | (turions) of aquatic | Xyländer, C., |
| phospphorylase | | vascular plant | Leeuwen, N. V., |
| | | greater duckweed | Schmidt, K-H and |
| | | (Spirodela | Appenroth, K-J. |
| | | polyrhiza) | 1999. J. Plant |
| | | | Physiol. 154: 37-45 |
| LEA genes | LEA gene | Douglas fir seeds | Jarvis, S. B., Taylor, |
| | expression affecting | (Pseudotsuga | M. A., Bianco, \grave{ } J., |
| | dormancy | menziesii (Mirb.) | Corbineau, F. and |
| | | Franco) | Davies, H. V. J. |
| | | | 1997 Plant Physiol. |
| | | | 151: 457-464 |
| Heat stable proteins | Heat stable proteins | Embryonic axes of | Ried, J. L. and |
| | responsive to ABA | dormant wheat | Walker-Simmons., |
| | | grains (Triticum | M. K. 1990. Plant |
| | | aestivum L. cv | Physiol. 93: 662- |
| | | Brevor) | 667. |
| CDNA clones | Differentially | Embryos of Avena | Johnson, R. R., |
| | expressed genes | fatua L. caryopses | Cranston, H. J., |
| | between dormant | | Chaverra, M. E. and |
| | and non-dormant | | Dyer, W. E. 1995. |
| | embryos | | Plant Mol. Biol. 28: |
| | | | 113-122 |
| Peroxiredoxin | AtPer1, a | Seeds of | Haslekas, C., Stacy, |
| antioxidant gene | peroxiredoxin | Arabidopsis thaliana | R. A. P., Nygaard, |
| | antioxidant gene is | C24 ecotype, | V., Culiáñez-Macià |
| | related to dormancy | landsberg (wild | and Aalen 1998. |
| | | type) and mutants, | Plant Mol. Biol. 36: |
| | | abi 3-1 and aba-1 | 833-845 |
| Antioxidant protein | PER1 peroxiredoxin, | Dormant seeds of | Stacy, R. A. P., |
| | antioxidant is related | Barley (Hordeum | Nordeng, T. W., |
| | to dormancy | vulgare L.) | Culiáñez-Macià, F. |
| | | | A. and Aalen, R. B. |
| | | | 1999. Plant J. 19: 1- |
| | | | 8. |
| Catalase | Catalase activity | One node cuttings of | Nir, G., Shulman, |
| | during dormancy | canes/or buds of | Y., Fanberstein, L. |
| | | Vitis vinifera L. | and Lavee, S. 1986. |
| | | | Plant Physiol. 81: |
| | | | 1140-1142 |
| Chilling and long | Chilling and long | Buds of Fagus | Heide. O. M. 1993. |
| days | days are required to | sylvatica | Physiol. Plant. 89: |
| | break dormancy | | 187-191 |
| α-amylase | Correlation of GA3 | Barley grains | Schuurink, R. C., |
| | responsiveness and | (Hordeum distichum | Sedee, N. J. A. and |
| | α-amylase secretion | L. cvs. Triumph and | Wang, M. 1992. |
| | with dormancy. | Kristina) | Plant physiol. 100: |
| | | | 1834-1839 |
| MRNA | Correlation of | Embryo/or whole | Goldmark, P. J., |
| | pBS128 transcript | seeds of Bromus | Curry, J., Morris, C. |
| | dormancy | secalinas (Cheat) | F. and Walker- |
| | phenomenon | frozen at −20° C. to | Simmons, M. K. |
| | | preserve dormancy | 1992. Plant Mol. |
| | | and in some | Biol. 19: 433-441 |
| | | dormancy was | |
| | | dissipated by post | |
| | | harvest storage | |
| p34 kinase | Cell cycle status and | Potato tubers stored | Campbell, M. A., |
| | p34^{cdc2} Kinase | at 3° C., transferred | Suttle, J. C. and Sell, |
| | expression during | to 20° C. after 120 | T. W. 1996. Physiol. |
| | dormancy | days and 223 days of | Plant. 98: 743-752 |
| | | storage | |
| GTP binding protein | GTP binding protein | Seeds of Fagus | Nicolás, C., Nicolás, |
| | related to dormancy | sylvatica | G. and Rodríguez, |
| | | | D. 1998. Plant Mol. |
| | | | Biol. 36: 487-491 |
| Cell cycle regulation | Accumulation of | Peas (Pisum sativum | Devitt, M. and |
| during growth and | mRNAs | L. cv. Alaska) were | Stafstrom, J. P. |
| dormancy | corresponding to | grown at 22° C. | 1995. Plant Mol. |
| | histones H2A and | under 16 h light/8 h | Biol. 29: 255-256 |
| | H4, ribosomal | dark photoperiod | |
| | protein genes rpL27 | | |
| | and rpL34, MAP | | |
| | kinase, cdc2 kinase | | |
| | and cyclin B were | | |
| | analysed during | | |
| | growth-dormancy | | |
| | cycles | | |
| SNF like protein | Up-regulation of | Buds of grapevines | Or, E., Vilozny, I., |
| kinase GDBRPK | GDBRPK a | (Vitis vinifera cv. | Eyal, Y. and |
| | transcript for SNF- | Perlette) | Ogrodovitch, A. |
| | like protein involved | | 2000. Plant Mol. |
| | in de-repression of | | Biol. 43: 483-494 |
| | meristemetic activity | | |
| | and release of | | |
| | dormanc | | |
| Synthesis of new | Seeds of Avena fatua | | Dyer, W. E. 1993. |
| proteins in the | L. line AN265 | | Physiol. Plant. 88: |
| embryos of imbibed | | | 201-211 |
| dormant seeds | | | |
| Inorganic | Related to non- | Hordeum cv. | Visser, K., |
| pyrophosphatase | dormancy | Triumph | Heimovaara- |
| | | | Dijkstra, Kijne, J. |
| | | | W. and Wang, |
| | | | M. 1998. Plant. Mol. |
| | | | Biol. 37: 131-140 |
| Peroxiredoxin | Related to dormancy | Seeds of barley | Stacy, R. A. P., |
| antioxidant. | and expressed | plants (Hordeum | Munthe, E., |
| | during late | vulgare L.). | Steinum, T., |
| | development in | | Sharma, B. and |
| | aleurone and embryo | | Aalen, R. B. 1996. |
| | of barley | | Plant. Mol. Biol. 31: |
| | | | 1205-1216 |
| MrnA and proteins | Changes mRNAs | Seeds of Trollius | Bailey, P. C., Lycett, |
| | and proteins during | ledebouri cv. Golden | G. W. and Roberts, |
| | dormancy and initial | Queen | J. A. 1996. Plant |
| | stages of | | Mol. Biol. 32: 559- |
| | germination | | 564 |
| Ca²⁺⁺ and | Altration in | Poplar plants | Jian, L-c., Li, P. H., |
| ultrastructure of | subcellular Ca²⁺⁺ | (Populus deltoides | Sun, L-h and Chen, |
| buds | localization and | Bartr. Ex | T. H. H. 1997. J. |
| | ultrastructure of | Marsh.) grown in | Exp. Bot. 48: 1195- |
| | apical bud cells | greenhouse | 1207 |
| | during development | conditions | |
| | of dormancy | | |
| Abscisic acid | Enhanced expression | Triticum aestivum L. | Morris, C. F., |
| reponsive genes | of five mRNAs in | cv Brevor seeds | Anderberg, R. J., |
| | relation to dormancy | | Goldmark, P. J. and |
| | | | Walker-Simmons, |
| | | | M. K. 1991. Plant |
| | | | Physiol. 95: 814-821 |
| ABA | ABA synthesized in | Two year old plants | Bris, M. L., |
| | the bud arrests cell | of Rosa hybrida L. | Michaux-Ferrière, |
| | cycle in G2 phase | cv. Ruidrilo Vivaldi | N., Jacob, Y., |
| | | | Poupet, A., Barthe, |
| | | | P., Guigonis, J-M. |
| | | | and Page-Degivry, |
| | | | M-T. L. 1999. Aust. |
| | | | J. Plant Physiol. 26: |
| | | | 273-281 |
| ABA | ABA is required for | Wild type tomato | Groot, S. P. C. and |
| | dormancy | (Lycopersicon | Karssen, C. M. |
| | maintenance | esculentum Mill. Cv | 1992. Plant Physiol. |
| | | Money maker) and | 99: 952-95 |
| | | isoganic ABA | |
| | | deficient line sit^{w} | |
| | | isogenic GA- | |
| | | deficient line gib-1 | |
| | | and isogenic | |
| | | recombinant line | |
| | | gib-1 sit^{w} | |
| Ribosomal protein | Negatively related to | Seedling of pea | Stafstrom, J. P. and |
| gene (pGB8) | dormancy | (Pisum sativum L. | Sussex, I. M. 1992. |
| | | cv Alaska) | Plant Physiol. 100: |
| | | | 1494-1502 |
| ABA | High level of ABA | Tuberose bulbs | Nagar, P. K. 1995. |
| | related to dormancy | (Polianthes tuberosa | Sci. Hort. 63: 77-82. |
| | | L.) | |
| Nucleic acids | Synthesis of nucleic | Tubers of Solanum | Macdonald, M. M. |
| | acids and proteins | tuberosum L. cv. | and Osborne, D. J. |
| | during dormancy | King Edward. | 1988 Physiol. Plant. |
| | and early sprouting | | 73: 392-400 |
| B15C cDNA and its | B15C Expression in | Anthers were | Aalen, R. B., |
| product. | aleurone layer and | cultured from the | Opsahl-Ferstad, H- |
| | embryo is related to | spikes of Hordeum | G., Linnestad, C. |
| | dormancy | vulgare cv. Dissa | and Olsen, O-A. |
| | | and perennial rye | 1994. Plant J. 5: |
| | | grass. Embryogenic | 385-396 |
| | | and non- | |
| | | embryogenic | |
| | | material separated | |
| | | under microscope | |
| Dehyrin and LEA | DF6 and DF77 | Seeds of Douglas fir | Jarvis, S. B., Taylor, |
| genes | cDNA clones related | (Pseudotsuga | M. A., Macleod, M. |
| | to dormancy | menziesii) | R., and Davies, H. |
| | | | V. 1996. J. Plant |
| | | | Physiol. 147: 559- |
| | | | 566 |
| Adenosine kinase | Enzyme activity | Flower and | Faye, F. and and |
| | affected by | vegetative buds | Floc'h, F. L. 1999. J. |
| | dormancy | peach tree (Prunus | Plant Physiol. 154: |
| | | persica L.) | 471-476 |
| Proteins | Effect of | Seeds of Doglas fir | Taylor, M. A., |
| | stratification on gene | (Pseudotsuga | Davies, H. V., |
| | expression during | menziesii) | Smith, S. B., |
| | dormancy breakage | | Abruzzese, A. and |
| | | | Gosling, P. G. 1993. |
| | | | J. Plant Physiol. 142: |
| | | | 120-123 |
| Carbohydrate | Relation between | Vegetative buds of | Marquat, C., |
| | carbohydrate | peach tree (Prunus | Vandamme, M., |
| | absorption potentials | persica L.) | Gendraud, M. and |
| | and carbohydrate | | Petél, G. 1999. Sci. |
| | concentration | | Hort. 79: 151-162 |
| Plant growth | Application of | Onion bulbs cv. Elba | Abdel-Rahman, M. |
| regulators | exogenous plant | | and Isenberg, F. M. |
| | growth regulators | | R. 1974. J. agric. |
| | modulate dormancy | | Sci. camb. 82: 113- |
| | | | 116 |
| Cell cycle specific | Chimeric β- | Buds of Populus | Rohde, A., Montagu, |
| promotor's activity | glucuronidase (gus) | (Populus tremula x | M. V. and Boerjan, |
| | genes under the | P. alba) | W. 1996. (In |
| | control of cell cycle | | Somatic Cell |
| | specific promotors | | Genetics and |
| | | | Molecular Genetics |
| | | | of Trees. eds Ahuja, |
| | | | M. R., Boerjan, W. |
| | | | and Neale, D. B. |
| | | | Kluwer Academic |
| | | | Publishers. |
| | | | Netherlands. pp 183- |
| | | | 188) |
| Promotor's activity | Activity of | Stem segments of | Nilson, O., Little, C. |
| | Agrobacterium | hybrid aspen | H. A., Sandberg, G. |
| | rizogenes rolC and | (Populusbtremula x | and Olsson, O. 1996. |
| | CaMV 35S | P. tremuloides) | Plant Mol. Biol. 31: |
| | promotor during | | 887-895 |
| | annual cycle of | | |
| | growth and | | |
| | dormancy | | |

There have been attempts to modulate dormancy process as follows:

(1) Bruce, D.; Eaton, C. D.; Schafer; Ronald, K. and Boise, I. D. in U.S. Pat. No. 5,811,372 dated Sep. 22, 1998 described a method to control the sprout formation in potatoes by prolonging the dormancy of potatoes during cold storage. Certain chemicals such as chloropham, carvone, bezothiazole were used with the average effective residue of CIPC (isopropyl 3—chlorophenyl-carbamate) on the tubers of approximately 16.6 ppm.

(2) Lulai; Edward, C. Orr; Paul, H., Glynn; Martin, T. in U.S. Pat. No. 5,436,226 dated Jul. 25, 1995 taught the suppression of sprouting of potato during storage in cool conditions by using jasmonates, a natural plant growth substance.

(3) Barry; Gerard Francis; Kishore; Ganesh Murthy; Stark; David Martin; Zalewaski; James Conrad in U.S. Pat. No. 5,648,249 dated Jul. 15, 1997 described a method of improving the quality of potatoes stored at reduced temperatures and a method of prolonging the dormancy of stored potato tubers. Transgenic potatoes were produced by transforming potato plants with a recombinant, double stranded DNA molecule comprising a promoter (cold-inducible promotor from potato or arabidopsis), a structural DNA sequence (which encoded a fusion polypeptide comprising an amino terminal plastid transit peptide and a E. Coli glgC ADPglucose pyrophosphorylase enzyme) and a 3′ end nontranslated DNA sequence (functiond to cause translational termination and the addition of polyadenylated nucleotides to the 3′ end of the RNA sequence). Promoter functioned to cause the production of an RNA sequence in tubers during cold storage and structural DNA sequence causes the production of an RNA Sequence. These transgenic potatoes tubers have been shown to exhibit prolonged dormancy period and inhibited sprouting at low temperature.

(4) Kawchuk, L. M.; Armstrong, J. D.; Lynch, D. R.; Knowles, N. R (1998) in Patent # WO9835051A1 dated Aug. 13, 1998 disclosed to improve the quality of potatoes stored at low temperatures by suppressing the gene expression. Inventor produced transgenic potatoes exhibiting reduced levels of glucan L-type phosphorylase (GLTP) or glucan H-type phosphorylase (GHTP) enzyme activity with in the potato tubers stored at low temperatures. The transgenic potatoes had prolonged dormancy and increased storage life.

(5) Khan, A. A. (1994) in U.S. Pat. No. 5,294,593 dated Mar. 15, 1994 discussed the procedure for introducing dormancy in non-dormant seeds of vegetables, grasses, trees and shrubs and flowers, by treating them with tetcyclasis an inhibitor of giberellin biosynthesis.

(6) Janes, H. W., Gore, G. E., Wittman, W. K., and Romen, H. T in U.S. Pat. No. 5,642,587 dated Jul. 1, 1997 disclosed the procedure to enhance the production and recovery of taxol and taxotrene by increasing the production of biomass. This was achieved by compressing the process of dormancy and by increasing the number of growing cycles within a greenhouse. To break dormancy, plants were placed in cold chamber under the conditions of no light and 85% of relative humidity for 30 days and then transferred to greenhouse (55 to 80° F. i.e., 13 and 27° C.; and relative humidity between 75% to 95%) for 40 day to grow. All the plants exposed to cold treatments showed more robust developed buds and overall plant growth. Untreated plants stopped growing and did not produce more growth after a period of time.

(7) Rieder, G. L. (U.S. Pat. No. 4,487,625 dated Dec. 11, 1984) discussed a method of interrupting bud dormancy in grapewine, a perennial crop, is described by spraying with 0.1 to 10 weight percent aquous cyanamide solution. Homogenous development of blossoms and fruits with increasing crop yield were obtained.

(8) Illingworth, J. (Dec. 2, 1997), in U.S. Pat. No. 5,693,592 disclosed certain compositions of growth regulating compounds (selected from the group consisting of fatty acid esters, fatty acid amides, fatty alcohols, fatty alcohol alkoxides, phthalate esters, phthalic acid amides and imides and mixture of two or more thereof) for controlling dormancy break and blooming in dormant perennial plants such as stone fruits (plums, cheeies, peaches etc), pome fruits (apples and peers), vines, grapes, olives, temperate fruits (Kiwi fruit, figs morus), berries (strawberries, raspberries, cranberies, blackberries, loganberries) and nuts (almonds, walnuts, chestnuts) which require a necessary cold stimulus to break bud dormancy. Varied concentrations (0.5 to 50% by weight) of different compositions of the above said growth regulating compounds were used in different plant species, in the dormant season with in 60 days before expected bud break. The invention was found to be useful to bring forward or setting back the time of bud break on fruit trees.

Below is specifically given a state of art knowledge with reference to the dormancy related genes from apical buds of the plants:

Reference may be made to document (1) by Or, E., Vilozny, I., Eyal, Y. and Ogrodovitch, A. 2000. Plant Mol. Biol. 43: 483-494, wherein is described the identification of a grape dormancy-breaking-related protein kinase (hereinafter referred to GDBRPK) transcript, which was up-regulated upon chemical induction of dormancy release by hydrogen cyanamide. GDBRPK has a 976 base pair (hereinafter known as, bp) open reading frame that encodes for 325 amino acids. This is followed by a 241 bp 3′-untranslated region. GDBRPK, presumably, served as a sensor of stress signal.

Reference may be made to document (2) by Rohde, A., Montagu, M. V. and Boerjan, W. 1996. (In Somatic Cell Genetics and Molecular Genetics of Trees. eds Ahuja, M. R., Boerjan, W. and Neale, D. B. Kluwer Academic Publishers. Netherlands. pp 183-188), wherein it was concluded that the process of bud and seed dormancy may share similar events. It may be noted that the work did not include any differential gene expression pattern or protein profiling studies. The conclusion was based upon work on transgenic Populus harboring chimeric β-glucuronidase gene under the control of cell cycle specific promoters. The studies, therefore, did not reveal any mechanism of bud dormancy per se or yielded any gene(s) related to the bud dormancy.

The drawbacks in the prior art are:

(a) Earlier efforts to modulate the dormancy by exposing the plants to low temperature condition is not possible for the plants standing in the field. Particularly, for the plants of, for example, Taxus, plums, peaches, apples, figs, morus, almonds, walnuts and tea etc., which being tree can not be grown in pots

(b) Earlier efforts to modulate the dormancy by spraying chemical formulations will not be environmental friendly and hence would contribute to environmental pollution

(c) There are no gene(s) till today, which have been cloned from dormant or the non-dormant buds under natural conditions. There is only one report on the isolation of the gene cloned from the buds of grapevine (Vitis vinifera cv. Perlette). However, the cloned gene is the one that is induced in response to the application of hydrogen cyanamide (Or, E., Vilozny, I., Eyal, Y. and Ogrodovitch, A. 2000. Plant Mol. Biol. 43: 483-494)

(d) In some of the cases, the gene of a particular enzyme has been taken from microorganism to be placed in the transgenic plant. This causes environmental concern and the hazard. The desirable feature would be to take the relevant gene from the plant system growing in similar ecosystem per se for the purpose of modulating the dormancy phenomenon

(e) There is no spectrum of the gene(s) expressed and repressed during the dormancy process

(f) The important aspect which has been not been considered in earlier invention is the same genetic make up of control and the treated tree species for the purpose of identification and cloning of the differentially expressed gene(s).

The above drawbacks have been eliminated for the first time in a simple and reliable manner by the present invention, which is not so obvious to the person skilled in the art.

### Benefits of the Invention

A benefit of the present invention is the cloning of novel DNA sequences expressed and repressed during winter dormancy in the apical buds of tea (Camellia sinensis L. (O.) Kuntze) bush growing under field conditions.

Yet another benefit of the present invention is to ensure the same genetic make up of the plant considered for the purpose of identification and cloning of novel DNA sequences expressed and repressed during winter dormancy in the apical buds of tea (Camellia sinensis L. (O.) Kuntze) bush growing under field conditions.

Yet another benefit of the present invention is to generate a spectrum of the gene(s) expressed and repressed during the process of winter dormancy in the apical buds of tea (Camellia sinensis L. (O.) Kuntze) bush growing under field conditions.

Yet another benefit of the present invention is the identification of 3′ ends of the gene(s) expressed and repressed during the process of winter dormancy in the apical buds of tea bush growing under field conditions.

Yet another benefit of the present invention is the confirmation of the identified 3′ ends of the differentially expressed gene(s) for establishing differential expression during winter dormancy in the apical buds of tea bush growing under field conditions.

Still another benefit of the present invention is the cloning of the identified 3′ ends of the differentially expressed gene(s).

Yet another benefit of the present invention is the sequencing of the identified 3′ ends of the cloned gene.

### BRIEF DESCRIPTION OF THE ACCOMPANYING DRAWINGS

FIG. 1 represents Total RNA isolated from the non-dormant (ND) and dormant (D) buds of tea. M represents RNA marker.

FIG. 2 represents cDNA synthesized from the total RNA isolated from the ND and D buds of tea. T₁₁A, T₁₁C and T₁₁G represent the primers used for the purpose of cDNA synthesis using total RNA in three separate reactions. M lane contains DNA molecular weight marker.

FIG. 3 represents spectrum of 3′ ends of the expressed and repressed genes in ND and D apical buds of tea using the primer combinations as defined at the bottom of each lane. Number on the top of each lane represents lane number. Arrow indicates differential expression.

FIG. 4 represents further spectrum of 3′ ends of the expressed and repressed genes in ND and D apical buds of tea using the primer combinations as defined at the bottom of each lane. Number on the top of each lane represents lane number. Arrow indicates differential expression.

FIG. 5 represents further spectrum of 3′ ends of the expressed and repressed genes in ND and D apical buds of tea using the primer combinations as defined at the bottom of each lane. Number on the top of each lane represents lane number. Arrow indicates differential expression.

FIG. 6 represents further spectrum of 3′ ends of the expressed and repressed genes in ND and D apical buds of tea using the primer combinations as defined at the bottom of each lane. Number on the top of each lane represents lane number. Arrow indicates differential expression.

FIG. 7 represents further spectrum of 3′ ends of the expressed and repressed genes in ND and D apical buds of tea using the primer combinations as defined at the bottom of each lane. Number on the top of each lane represents lane number. Arrow indicates differential expression.

FIG. 8 represents further spectrum of 3′ ends of the expressed and repressed genes in ND and D apical buds of tea using the primer combinations as defined at the bottom of each lane. Number on the top of each lane represents lane number. Arrow indicates differential expression.

FIG. 9 represents further spectrum of 3′ ends of the expressed and repressed genes in ND and D apical buds of tea using the primer combinations as defined at the bottom of each lane. Number on the top of each lane represents lane number. Arrow indicates differential expression.

FIG. 10 represents further spectrum of 3′ ends of the expressed and repressed genes in ND and D apical buds of tea using the primer combinations as defined at the bottom of each lane. Number on the top of each lane represents lane number. Arrow indicates differential expression.

FIG. 11 represents amplification of the differentially expressed 3′ ends of the gene after eluting from the sequencing (denaturing polyacrylamide) gels as shown in FIGS. 3-10. The first number at the top of each lane represents the lane number as mentioned in FIGS. 3-10. The second number followed by the dot represents the number of differentially expressed band as counted from the top of the respective lane as mentioned in FIGS. 3-10. M represents DNA size marker.

FIG. 12 represents further amplification of the differentially expressed 3′ ends of the gene after eluting from the sequencing (denaturing polyacrylamide) gels as shown in FIGS. 3-10. The first number at the top of each lane represents the lane number as mentioned in FIGS. 3-10. The second number followed by the dot represents the number of differentially expressed band as counted from the top of the respective lane as mentioned in FIGS. 3-10. M represents DNA size marker.

FIG. 13 represents further amplification of the differentially expressed 3′ ends of the gene after eluting from the sequencing (denaturing polyacrylamide) gels as shown in FIGS. 3-10. The first number at the top of each lane represents the lane number as mentioned in FIGS. 3-10. The second number followed by the dot represents the number of differentially expressed band as counted from the top of the respective lane as mentioned in FIGS. 3-10. M represents DNA size marker.

FIG. 14 represents further amplification of the differentially expressed 3′ ends of the gene after eluting from the sequencing (denaturing polyacrylamide) gels as shown in FIGS. 3-10. The first number at the top of each lane represents the lane number as mentioned in FIGS. 3-10. The second number followed by the dot represents the number of differentially expressed band as counted from the top of the respective lane as mentioned in FIGS. 3-10. M represents DNA size marker.

FIG. 15 represents amplification after cloning of the eluted differentially expressed 3′ ends of the gene as mentioned in FIGS. 11-14. The first number at the top of each lane represents the lane number as mentioned in FIGS. 3-10. The second number followed by the dot represents the number of differentially expressed band as counted from the top of the respective lane as mentioned in FIGS. 3-10. M represents DNA size marker.

FIG. 16 represents amplification after cloning of the eluted differentially expressed 3′ ends of the gene as mentioned in FIGS. 11-14. The first number at the top of each lane represents the lane number as mentioned in FIGS. 3-10. The second number followed by the dot represents the number of differentially expressed band as counted from the top of the respective lane as mentioned in FIGS. 3-10. M represents DNA size marker.

FIG. 17 represents amplification after cloning of the eluted differentially expressed 3′ ends of the gene as mentioned in FIGS. 11-14. The first number at the top of each lane represents the lane number as mentioned in FIGS. 3-10. The second number followed by the dot represents the number of differentially expressed band as counted from the top of the respective lane as mentioned in FIGS. 3-10. M represents DNA size marker.

FIG. 18 represents confirmation of differential expression of the cloned 3′ ends of the gene through northern hybridization.

FIG. 19 represents further confirmation of differential expression of the cloned 3′ ends of the gene through northern hybridization using 2 more clones.

FIG. 20 represents expression of the identified, cloned 3′ ends of gene number 31.2 from ND apical buds in ND, D and forced ND apical buds (gibberellic acid, GA₃, was applied onto the D buds during winter season to force the buds to enter into non-dormancy stage).

### SUMMARY OF THE INVENTION

Accordingly, the present invention provides:

(a) novel DNA sequences expressed and repressed during winter dormancy in the apical buds of Camellia sinensis L. (O.) Kuntze (tea) bush under field conditions,

(b) cloning of such sequences have been from the same genetic make up of the tea bush,

(c) a spectrum of 3′ ends of the expressed and repressed genes in non-dormant and dormant apical buds of tea bush growing under field conditions,

(d) confirmation of the identified 3′ ends of the differentially expressed gene(s) for establishing differential expression during winter dormancy in the apical buds of tea bush growing under field conditions,

(e) method to correlate the identified gene with the dormancy of tea buds under field conditions, and

(f) sequencing of the cloned 3′ ends of the differentially expressed gene(s) showed uniqueness in terms of novel sequences not deposited in the data bank so far.

### DETAILED DESCRIPTION OF THE INVENTIONS

Accordingly, the present invention provides novel DNA sequences expressed and repressed during winter dormancy in the apical buds of Camellia sinensis L. (O.) Kuntze (tea) bush or a tree species, said sequences comprising sequence ID 1 to 4 as shown here below:

SEQ ID NO: 1

5′-ATCGCCGTAA TTGCCATGTT TTCCCTCTCA CCGGAATCCT ACG
TTATCC CCTTACCTTC GTGAACATTA CAGTAGGAAT CGGTGGTCCA
ATTATCAACT TAATTTTGGG CGCATCTGTT CGTGTTAACT
AGAAGCCATG TATACATACA ATACAACATG GTTCACTCCT
CCTACAGATT ATGAGTTGAA CTTTTATAAT AAGTTGTAAT
AATGGCTTCT GAATAAGGAG AAGAGGAGCC TCTGTTTGTT
TTACTTATTA CAGATGTGAT ATCGTTCAAC AACTTTGATT
CTGCGAAAAA AAAAA-3′

SEQ ID NO: 2

5′-AGAAGTACCT GAAAGGAAGC TTAACGAGGT GAACATCCAT
TGCAGCCAGC CCTGGAATCT GTACAGGGCA ACTCTGAACC
GGAATTATTT TAATAACCCG TGGGCAATGA TTGCAATTAT
GGCTCGTTTG GTATTACTTC TACTCACTTA GACACAACTG
TATTTACGGT TTTCGCTGGA ATTGTAATTG TTGGAGCGAC
AAAATAGATG GTCACAACTT ATTGGTGAGA GTATCAGTGT
GCTCTTCTTT ATCGTCTTTA ACTCTCCGTG GTAATTACTT
TGACAATATT CATACAT-3′

SEQ ID NO: 3

5′-GAGACTCAGC TCAGCAATCA TGTTCTAAGT GAATGTCACT
CTATCGCCTT CTTGTCCCTC TTAGACATAC TACATCCTCA
TTCTGCTAGA AATGAACTCA TGTAGGTTTT GAAGTTGGGA
ACTTTTGAAA CTGTGTTGTT TGGGTGCTGT CTGTTATACA
ATTCTCTCAA CTGCGGAGAA TTGACGTTGG TTGTAGTGGA
ATTCAACACT TGGGTTTTGT TCTTAGTTAA AAAAAAAAA-3′

SEQ ID NO: 4

5′-ATAGCTTAGT CACGTGTCTC TTGAGAATGG ACTACGTAGT
TGTTAAGTTG GGTGATCAGA AGGCGTTGAT GATGAATGTA
TGAAGCAGAG ACTACTGAAT GTAATTTTGT TGTTGAAAGA
TGAATGATTT ATTAATGCCT GCATATCTTT CTATTGTTTG
ATGCCAAACC TTTGGGCACA TTTTTTCTTT CTTTTTGTGA
TAATGTTCTC TTCTTGCAAA AAAAAAAA-3′

In an embodiment of the invention, the novel DNA sequences, which are cloned from the tea bush of the same, genetic make up.

In another embodiment of the invention, the novel DNA sequences, which are cloned from the tea bush of the same genetic make up growing under field conditions.

In another Novel DNA sequences as claimed in claim 1, are associated with winter dormancy in tea.

In still another embodiment of the invention, the novel DNA sequences, which are cloned by any methods but not limited to, subtractive hybridization and differential screening.

In yet another embodiment of the invention, the nucleotide sequence of the DNA is given in SEQ ID NO 1.

In yet another embodiment of the invention, the nucleotide sequence of the DNA as is given in SEQ ID NO 1 is overexpressed only in non-dormant apical buds of tea.

In yet another embodiment of the invention, the nucleotide sequence of the DNA is given in SEQ ID NO 2

In yet another embodiment of the invention, the nucleotide sequence of the DNA as is given in SEQ ID NO 2 is expressed only in non-dormant apical buds of tea.

In yet another embodiment of the invention, the nucleotide sequence of the DNA is given in SEQ ID NO: 3

In yet another embodiment of the invention, the nucleotide sequence of the DNA as is given in SEQ ID NO 3 is expressed only in non-dormant apical buds of tea.

In yet another embodiment of the invention, the nucleotide sequence of the DNA is given in SEQ ID NO: 4

In yet another embodiment of the invention, the nucleotide sequence of the DNA as is given in SEQ ID NO 4 is expressed only in dormant apical buds of tea.

In yet another embodiment of the invention, the novel sequences are capable of being cloned to full-length cDNA.

In yet another embodiment of the invention, the novel sequences are capable of being cloned to full length genomic DNA.

In yet another embodiment of the invention, the novel sequences are capable of being cloned to important sequences, such as but not limited to, promoter sequences and regulatory sequences etc.,

Tea bushes, belonging to chinary type, growing and maintained in the tea farm of the Institute of Himalayan Bioresource Technology, Palampur (32° 04′ N, 76° 29′ E; altitude, 1300 m) were selected. Tea plants belonging to other types namely, assamica and combod could have also been selected. However, due to predominance of the chinary type in Kangra region of the Himachal Pradesh, this particular clone was selected.

In a preferred embodiment of the present invention the genetic make of the plant, considered for the purpose of identification and cloning of novel DNA sequences expressed and repressed during winter dormancy in the apical buds of tea (Camellia sinensis L. (O.) Kuntze) bush growing under field conditions was ensured to be the same. This is important for a plant like tea. Tea is a highly cross-pollinated plant and grown by the seed. This leads to enormous plant to plant heterogeneity due to differential genetic make up. Each seed raised plant of tea is regarded a clone in itself. Particularly, for the inventions related to compare gene expression pattern for the purpose of cloning the differentially expressed gene in response to an altered environmental condition, such as is the purpose of the present invention of comparing and cloning the expressed and repressed gene(s) from the control and a winter dormant plant, it is absolutely essential that the plants experiencing altered environmental conditions have the same genetic make up. Having realized this important aspect, only one bush of tea belonging to chinary type was selected.

In another embodiment of the present invention the apical buds of tea measuring 1.2-1.5 cm (in length); 0.5-0.6 cm (perimeter) and were collected during non-dormant season (in the month of April) at 10.00 O'clock in the morning from only one bush. Apical buds during non-dormant season grow to open into the leaves. These are referred to as non-dormant (hereinafter known as ND) buds and are considered “control” within the scope of the present invention. The bush yielded around 100 numbers of apical buds. Apical buds were washed with diethyl pyrocarbonate (hereinafter known as DEPC) treated water [to prepare DEPC treated water, DEPC was added in distilled water to a final concentration of 0.1% followed by autoclaving (i.e. heating at 121° C. under a pressure of 1.1 kg per square centimeters) after an overnight incubation.], harvested and immediately dipped in liquid nitrogen to freeze the cellular constituents for ceasing the cellular activities.

Tea bush became dormant from the month of November onwards. The apical buds during this period do not grow in the size to open into the leaves and are referred to as dormant (hereinafter known as D) buds. The same bush, from which the ND buds were collected, was used to collect the D buds. D buds, measuring the similar dimensions as for the ND buds, were plucked and stored essentially as described for ND buds.

Collection of ND and D buds from the same bush ensured the same genetic make up of the tissue under consideration.

In another embodiment of the present invention total RNA from ND and D buds was isolated and the “differential display technique” (Liang, P., Zhu, W., Zhang, X., Guo, Z., O'Connell, R., Averboukh, L., Wang, F. and Pardee, A. B. 1994. Nucleic acids Res. 22: 1385-1386) was employed to generate a spectrum of 3′ ends of the expressed and repressed genes in ND and D buds of tea

In an advantageous embodiment of the present invention 3′ ends of the expressed and repressed genes in ND and D buds of tea were ligated into a vector to yield a recombinant plasmid, which upon transformation into a suitable E. coli host resulted into a clone. Vector, in the present invention refers to the sequence of DNA capable of accepting foreign DNA and take the form of a circular plasmid DNA that shows resistance to a given antibiotic.

In yet another embodiment of the present invention the gene cloned was tested for its expression or repression in ND and D buds of tea to define association of the cloned gene with the dormancy process.

In another embodiment of the present invention the gene was sequenced using the dideoxy chain termination method (Sanger, F. S., Nicklen, S. and Coulson, A. R. 1977. Proc. Natl. Acad. Sci. USA. 74: 5463-5467) to figure out the uniqueness of the gene.

One more embodiment of the invention, wherein the sequence data is used for obtaining, important information on the gene regulation.

In another embodiment of the invention, the novel genes are used to modulate winter dormancy in plants after transferring these genes using the techniques such as, but not limited to, Agrobacterium mediated transformation and Biallistic medited transformation

In another embodiment of the invention, it is possible to modulate winter dormancy using the novel genes in the plants such as, but not limited to, tea, plums, cherries, peaches, Taxus, apples, peers, vines, grapes, olives, Kiwi fruit, figs, morus, strawberries, raspberries, cranberies, blackberries, loganberries, almonds, walnuts and chestnuts after transferring these genes using the techniques such as, but not limited to, Agrobacterium mediated transformation and bialistic medited transformation.

In still another embodiment of the invention, use of sequence data of the novel genes for obtaining important information on the gene regulation to be exploited to regulate gene expression in transgene.

In still another embodiment of the invention relates to use of cDNAs and the genomic DNAs for synthesizing unique proteins and in addition use of unique proteins for raising antibodies.

In yet another embodiment of the invention relates to use of the present antibodies as probe to look for the similar proteins in other plants, animal and/or microbial systems or the like.

In yet another embodiment of the invention relates to use of novel sequences, cDNAs and the genomic DNAs of the present invention as probe to look for the sequences of nucleotides in other plants, animal and/or microbial systems and the like.

In yet another embodiment of the invention relates to use of novel sequences, cDNAs and the genomic DNAs of the present invention, as probe to look for the expression of these sequences of nucleotides in other plants, animal and/or microbial systems and the like.

In yet another embodiment of the invention relates a method to correlate the identified gene with the process of dormancy of tea buds as described for sequence ID 1 is unique.

In yet another embodiment of the invention relates a method which, can be applied to other sequence ID as well.

In yet another embodiment of the invention relates to a method which, can be applied to other crops such as, but not limited to plums, cherries, peaches, Taxus, apples, peers, vines, grapes, olives, Kiwi fruit, figs, morus, strawberries, raspberries, cranberries, blackberries, loganberries, almonds, walnuts and chestnuts as well for correlating similar genes.

The present invention will be illustrated in greater details by the following examples. These examples are presented for illustrative purposes only and should not be construed as limiting the invention, which is properly delineated in the claims.

### EXAMPLE 1

RNA Isolation, Digestion of RNA with DNase 1, Quantification of RNA and Gel-Electrophoresis:

To ensure a high quality of ribonucleic acid (hereinafter known as, RNA) from ND and D buds of tea bush, RNeasy plant mini kits (purchased from M/s. Qiagen, Germany) were used. Manufacturer's instructions were followed to isolate RNA. RNA was quantified by measuring absorbance at 260 nm and the purity was monitored by calculating the ratio of absorbance meaasured at 260 and 280 nm. A value >1.8 at 260/280 nm was considered ideal for the purpose of present investigation. The formula used to calculate RNA concentration and yield was as follows:

| | |
|---|---|
| Concentration of RNA (μg/ml) = | A₂₆₀ (absorbance at 260 nm) × 40 × |
| | dilution factor |
| Total yield (μg) = | concentration × volume of stock RNA |
| | sample |

To check the intigrity of RNA, 5-6 μg of RNA in 4.5 μl was diluted with 15.5 μl of M1 solution (2 μl of 5×MOPS buffer, 3.5 μl of formaldehyde, and 10 μl of formamide [5×MOPS buffer: 300 mM sodium acetate, 10 mM MOPS (3-{N-morpholino]propanesulfonic acid}, 0.5 mM ethylene diamine tetra-acetic acid (EDTA)]and incubated for 15 minutes at 65° C. RNA was loaded onto 1.5% formaldehyde agarose-gel after adding 2 μl of formaldehyde-gel loading buffer [50% glycerol, 1 mM EDTA (pH, 8.0), 0.25% bromophenol blue, 0.25% xylene cyanol FF], and electrophoresed at 72 volts in 1×MOPS buffer (60 mM sodium acetate, 2 mM MOPS, 0.1 mM EDTA), (Sambrook, J., Fritsch, E. F. and Maniatis, T. 1989. Molecular Cloning: A Laboratory Manual, Cold Spring Harbor Laboratory Press, Plainview, N.Y.).

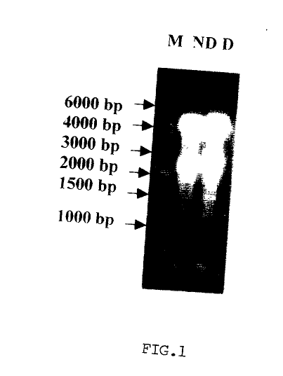

To remove the residual DNA, RNA (10-50 μg) was digested using 10 units of DNase I, in 1×reaction buffer [10×reaction buffer: 100 mM Tris-Cl (pH, 8.4), 500 mM KCl, 15 mM MgCl₂, 0.01% gelatin] at 37° C. for 30 minutes (Message clean kit from M's. GenHunter Corporation, USA). DNase I was precipitated by adding PCI (phenol, chloroform, isoamylalcohol in ratio of 25:24:1) and RNA present in the aqueous phase was precipitated by adding 3 volumes of ethanol in the presence of 0.3 M sodium acetate. After incubating for 3 hours at −70° C., RNA was pelleted, rinsed with chilled 70% ethanol and finally dissolved in 10 μl of RNase free water. DNA-free-RNA thus obtained was quantified and the integrity was checked as above. The quality of RNA is depicted in FIG. 1. Although we have used Rneasy colmns from M/S Quiagen, Germany, the other procedure can also be used to isolate from the apical buds of tea.

### EXAMPLE 2

Conversion of mRNA into Complementary DNAs (Hereinafter Referred to cDNAs) by Reverse Transcription (Hereinafter Referred to RT):

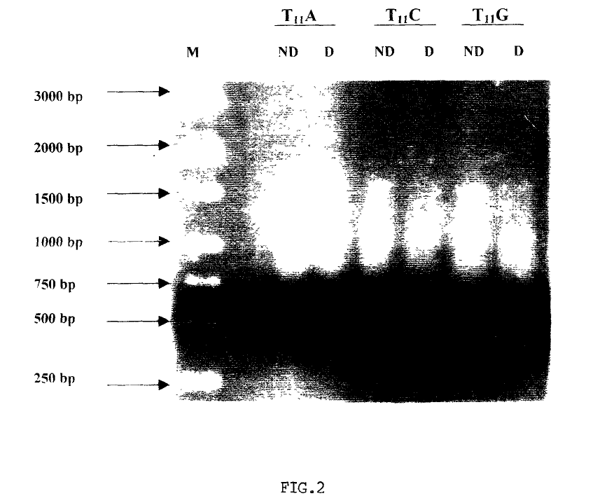

0.2 μg of DNA-free-RNA from dormant and non-dormant samples was reverse transcribed in separate reactions to yield cDNAs using an enzyme known as reverse transcriptase. The reaction was carried out using 0.2 μM of T₁₁M primers (M in T₁₁M could be either T₁₁A, T₁₁C or T₁₁G), 20 μM of dNTPs, RNA and RT buffer [25 mM Tris-Cl (pH, 8.3), 37.6 mM KCl, 1.5 mM MgCl₂ and 5 mM DTT]. In the present invention dNTP refers to deoxyadenosine triphosphate (hereinafter reffered to dATP), deoxyguanosine triphosphate (hereinafter reffered to dGTP), deoxycytidine triphosphate (herein after reffered to dCTP) and deoxythymidine triphosphate (hereinafter reffered to dTTP). Three RT reactions were set per RNA sample for the corresponding T₁₁M primer. The reactions were carried out in a thermocycler (model 480 from M/s Perkin-Elmer). Thermocycler parameters chosen for transcription were 65° C. for 5 minutes, →37° C. for 60 minutes, →75° C. for 5 minutes, →4° C. 100 units of reverse transcriptase was added to each reaction after 10 minute incubation at 37° C. and reaction then continued for rest of the 50 minutes. This yielded cDNAs as is shown in FIG. 2. Two different RNA (ND and D) in combination with 3 T₁₁M primers yielded a total of 6 reactions depicting 6 different classes of cDNAs. The use of 3 different T₁₁M primers divided the whole RNA population into 3 sub-classes depending upon the anchored base M, which was either A, C or G (Reverse transcription system was a component of RNAimage kit from M's. GenHunter Corporation, USA).

### EXAMPLE 3

Generation of a Spectrum of Differentially Expressed Genes Through Differential Display Technique: Identification of Differentially Expressed Gene(s):

Different sub-classes of cDNA from dormant and non-dormant RT product as obtained in Example 2 were amplified in the presence of a radiolabelled dNTP to label the amplified product through polymerase chain reaction (hereinafter known as PCR; PCR process is covered by patents owned by Hoffman-La Roche Inc.). Radioactive PCR was carried out in 20 μl reaction mix containing a (1) reaction buffer [10 mM Tris-Cl (pH, 8.4), 50 mM KCl, 1.5 mM MgCl₂, 0.001% gelatin], (2) 2 μM dNTPs, (3) 0.2 μM T₁₁M and (4) 0.2 μM arbitrary primers (chemicals 1 to 4 were purchased from M/s. GenHunter Corporation, Nashville, USA as a part of RNAimage kit), 0.2 μl α[³³P] dATP (˜2000 Ci/mmole, purchased from JONAKI Center, CCMB campus Hyderabad, India), and 1.0 units of Thermus aqueticus (hereinafter referred to Taq) DNA Polymerase (purchased from M/S. Qiagen, Germany). 30 μl of autoclaved mineral oil was overlaid at the top of each reaction to avoid alteration in volume due to evaporation. T₁₁M primer in each reaction was the same that was used to synthesize cDNA. Parameters choosen were: 40 cycles of 94° C. for 30 seconds,→40° C. for 2 minutes,→72° C. for 30 seconds; and 1 cycle of 72° C. for 5 minutes and final incubation at 4° C.

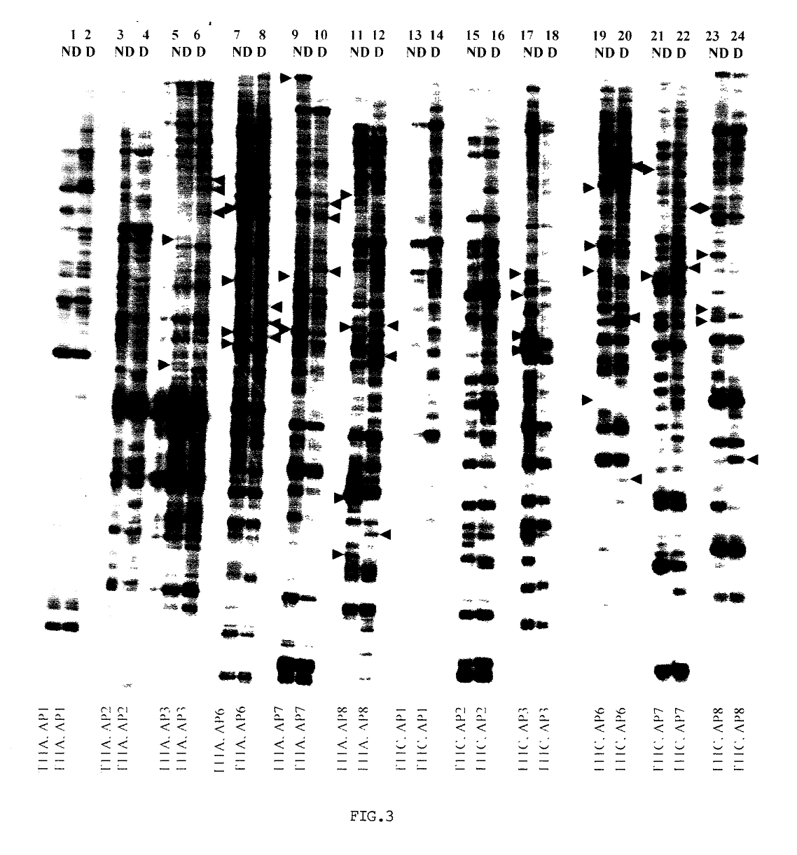

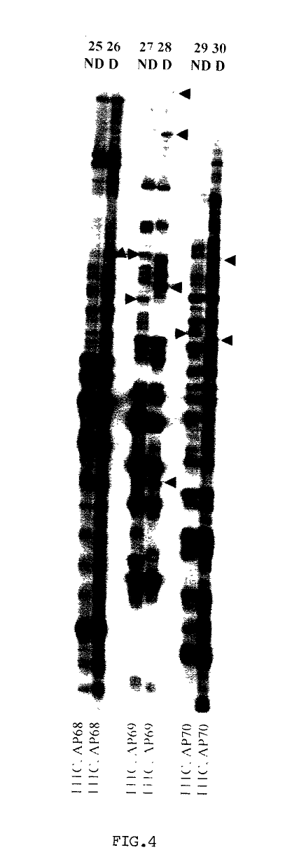

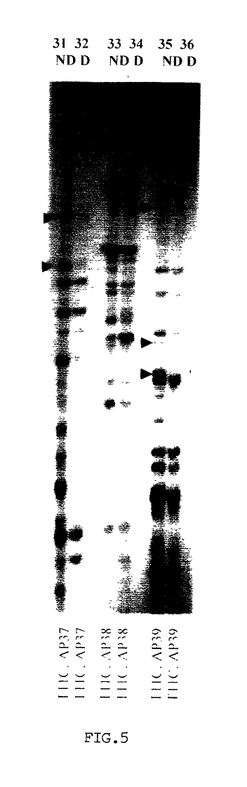

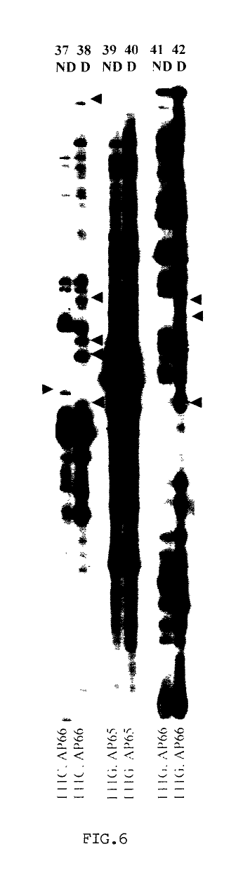

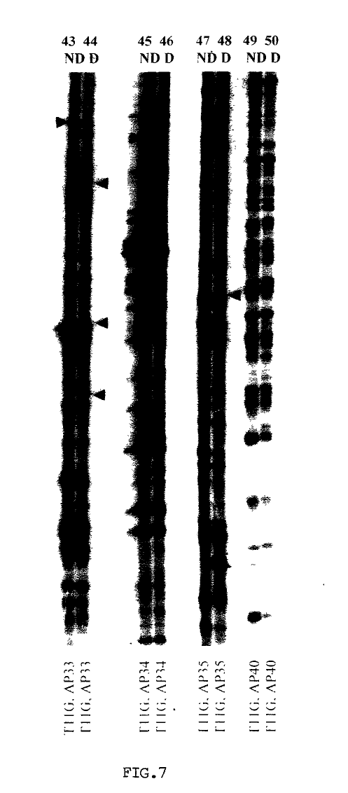

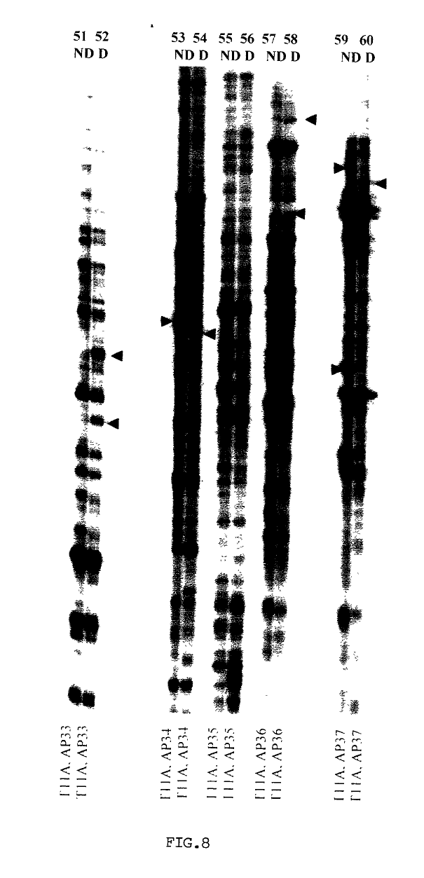

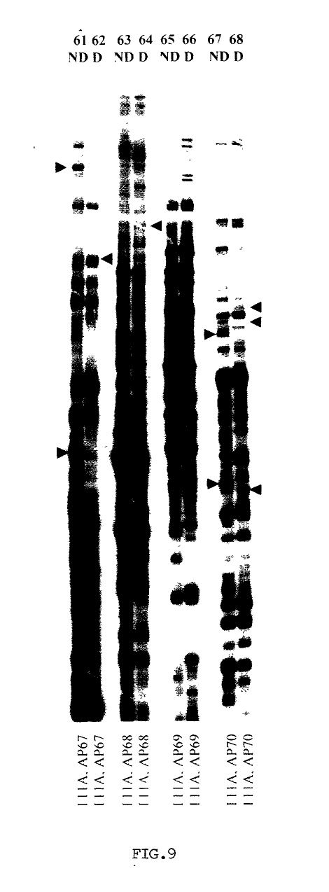

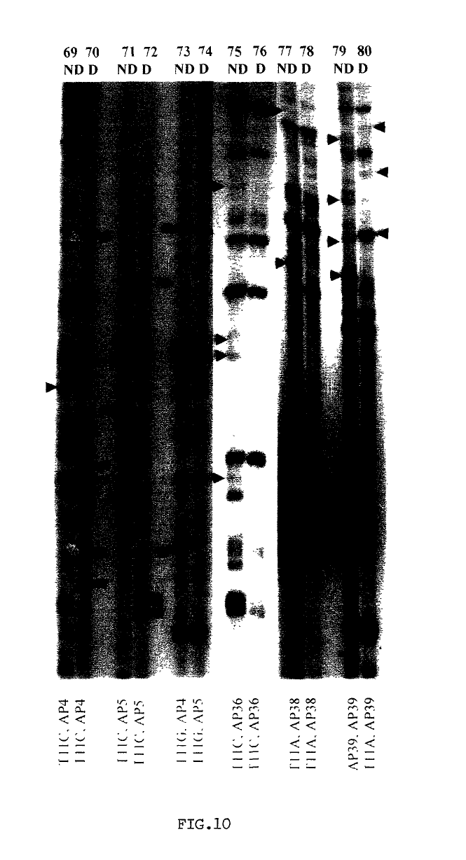

Amplified products were fractionated onto a 6% denaturating polyacrlamide gel. For the purpose 3.5 μl of each of amplified product was mixed with 2 μl of loading dye [95% formamide, 10 mM EDTA (pH, 8.0), 0.09% xylene cyanol FF and 0.09% bromophenol blue], incubated at 80° C. for 2 minutes and loaded onto a 6% denaturating polyacrlamide gel [denaturating polyacrlamide gel: 15 ml of acrylamide (40% stock of acrylamide and bisacrylamide in the ratio of 20:1), 10 ml of 10×TBE, 40 ml of distilled water and 50 g urea]. Electrophoresis was performed using 1×TBE buffer [10×TBE: 108 g Tris base, 55 g boric acid and 40 ml of 0.5 M EDTA (pH, 8.0)] as a running buffer at 60 watts until the xylene cyanol (the slower moving dye) reached the lower end of the glass plates. Size of the larger plate of the sequencing (denaturing polyacylamide) gel apparatus was 13×16 inch. After the electrophoresis, one of the glass plates was removed and the gel was transferred onto a 3 MM Whattman filter paper. Gel was dried at 80° C. overnight and exposed to Kodak X-ray film for 2-3 days. Before exposing to X-ray film, corners of the dried gel were marked with radioactive ink for further alignment. FIGS. 3 to 11 show the spectrum of differentially expressed genes in ND and D apical buds of tea as was seen after developing the film. After developing the gel, film was analyzed for differential expressed bands between ND and D signals.

Sequences of the Primers Used for Differential Display were as Follows (Purchased from M/s. GenHunter Corporation, USA as a prt of RNA Image Kit):

Primer sequence

T₁₁M (anchored) primers

| | |
|---|---|
| T₁₁A | 5′-AAGCTTTTTTTTTTTTTA-3′ |
| T₁₁C | 5′-AAGCTTTTTTTTTTTTTC-3′ |
| T₁₁G | 5′-AAGCTTTTTTTTTTTTTG-3′ |

Arbitrary Primers

| | |
|---|---|
| AP1 | 5′-AAGCTTGATTGCC-3′ |
| AP2 | 5′-AAGCTTCGACTGT-3′ |
| AP3 | 5′-AAGCTTTGGTCAG-3′ |
| AP4 | 5′-AAGCTTCTCAACG-3′ |
| AP5 | 5′-AAGCTTAGTAGGC-3′ |
| AP6 | 5′-AAGCTTGCACCAT-3′ |
| AP7 | 5′-AAGCTTAACGAGG-3′ |
| AP8 | 5′-AAGCTTTTACCGC-3′ |
| AP33 | 5′-AAGCTTGCTGCTC-3′ |
| AP34 | 5′-AAGCTTCAGCAGC-3′ |
| AP35 | 5′-AAGCTTCAGGGCA-3′ |
| AP36 | 5′-AAGCTTCGACGCT-3′ |
| AP37 | 5′-AAGCTTGGGCCTA-3′ |
| AP38 | 5′-AAGCTTCCAGTGC-3′ |
| AP39 | 5′-AAGCTTTCCCAGC-3′ |
| AP40 | 5′-AAGCTTGTCAGCC-3′ |
| AP65 | 5′-AAGCTT CAAGACC-3′ |
| AP66 | 5′-AAGCTT GCCTTTA-3′ |
| AP67 | 5′-AAGCTT TATTTAT-3′ |
| AP68 | 5′-AAGCTT CTTTGGT-3′ |
| AP69 | 5′-AAGCTT AATAACG-3′ |
| AP70 | 5′-AAGCTT TCATATG-3′ |
| AP71 | 5′-AAGCTT GTAGTAA-3′ |
| AP72 | 5′-AAGCTT TCAAAGA-3′ |

### EXAMPLE 4

Reamplification of cDNA Probes:

Cloning the differentially expressed bands required elution of the same from the denaturating polyacrylamide gel and further amplification to yield substantial quantity of DNA for the purpose of cloning. Autoradiogram (developed X-ray film) was oriented with the dried gel aided with radioactive ink and the identified differentially expressed band (along with the gel and the filter paper) was cut with the help of a sterile sharp razor. DNA was eluted by incubating in 100 μl of sterile dH₂O for 10 min in an eppendorf tube, followed by boiling for 10 minutes. Paper and gel debris were pelleted by spinning at 10,000 rpm for 2 min and the supernatant containing DNA was transferred into a new tube. DNA was precipitated with 10 μl of 3M sodium acetate, pH,5.5, 5 μl of glycogen (stock; 10 mg/ml) and 450 μl of ethanol. After incubation at −70° C. for overnight, centrifugation was performed at 10, 000 rpm for 10 min at 4° C. and pelleted DNA was rinsed with 85% ethanol. DNA pellet was dissolved in 10 μl of sterile distilled water.

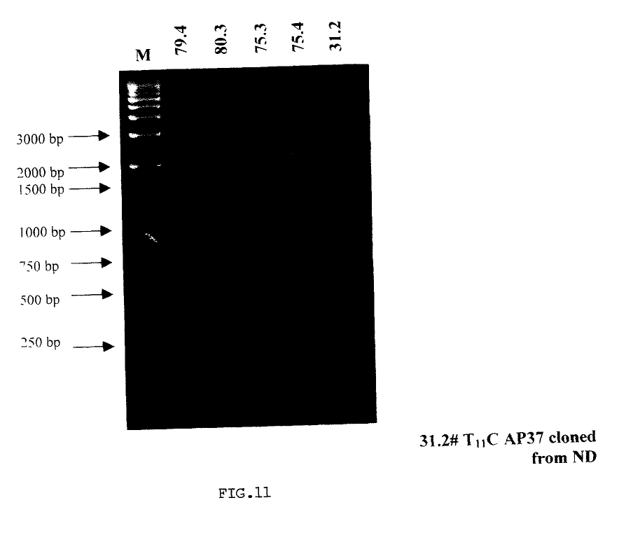

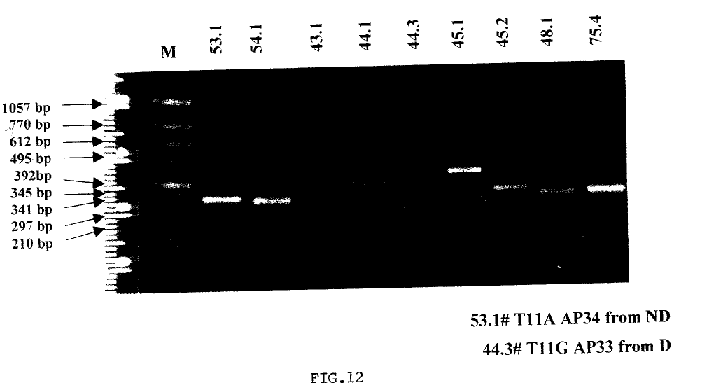

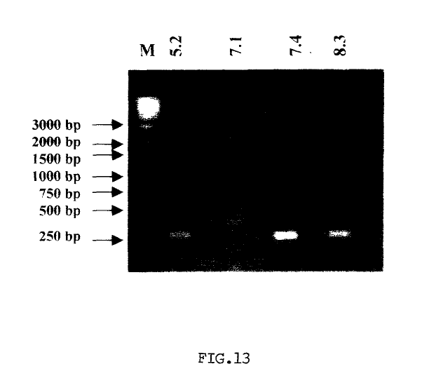

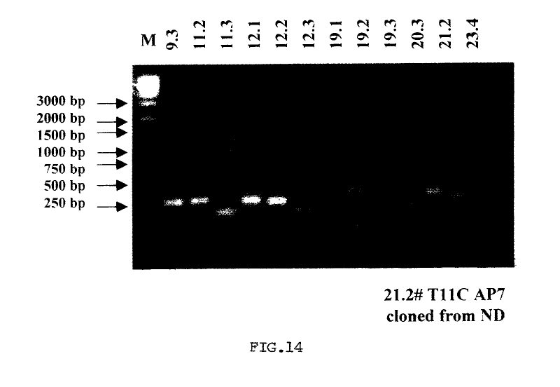

Eluted DNA was amplified using the same set of T₁₁M and arbitrary primer that was used for the purpose of performing differential display as in the Example 3. Also, the PCR conditions were the same except that dNTP concentration was 20 μM instead of 2 μM and no isotopes was added. Reaction was up-scaled to 40 μl and after completion of PCR, 30 μl of PCR sample was run on 1.5% agarose gel in TAE buffer (TAE buffer: 0.04 M Tris-acetate, 0.002 M EDTA, pH 8.5) containing ethidium bromide (final concentration of 0.5 μg/ml). Rest of the amplified product was stored at −20° C. for cloning purposes (see FIGS. 11-14).

### EXAMPLE 5

Cloning of Re-Amplified PCR Products:

Re-amplified PCR products as obtained in example 4 were ligated in 300 ng of insert-ready vector called as PCR-TRAP® vector using 200 units of T4 DNA-ligase in 1×ligation buffer (10×ligase buffer: 500 mM Tris-Cl, pH 7.8, 100 mM MgCl2, 100 mM DTT, 10 mM ATP, 500 μg/ml BSA). Vector and the other chemicals required were purchased from M/s. GenHunter Corporation, Nashville, USA as PCR-TRAP® cloning system. Ligation was performed at 16° C. for 16 hours in a thermocycler model 480 from M's. Perkin Elmer, USA. Ligation of the PCR product into a vector such as above yields to a circularized plasmid. The process of ligation of the foreign DNA, such as the PCR product in the present invention, into a suitable vector, such as PCR-TRAP® vector in the present invention, is known as cloning. There is a range of other vectors that are commercially available or otherwise that suits the cloning work of PCR products and hence may be used. The plasmid, as per the definition, is a closed circular DNA molecules that exists in a suitable host cell such as in Escsherichia coli (hereinafter referred to E. coli) independent of chromosomal DNA and may confer resistance against an antibiotic. PCR-TRAP® vector resulting plasmid confers resistance against tetracycline.

Ligated product or the plasmid needs to be placed in a suitable E. coli host for its multiplication and propagation through a process called transformation. Ligated product (10 μl) as obtained above was used to transform 100 μl of competent E. coli cells (purchased from M/s. GenHunter Corporation USA as a part of PCR-TRAP cloning system). Competent means the E. coli cells capable of accepting a plasmid DNA. For the purpose, ligated product and competent cell were mixed, kept on ice for 45 minutes, heat shocked for 2 minutes and cultured in 0.4 ml of LB medium (LB: for 1 litre: 10 g tryptone, 5 g yeast extract, 10 g sodium chloride) for 4 hours. 200 μl of transformed cells were plated onto LB-tetracyclin (for 1 litre: 10 g tryptone, 5 g yeast extract, 10 g sodium chloride, and tetracyclin added to a final concentration of 20 μg/ml) plates and grown overnight at 37° C. Colonies were marked and single isolated colonies were restreaked on to LB-tetracyclin plates to get colonies of the same kind. Conferral of tetracyclin resistance to E. coli cells apparently suggests that the PCR product i.e. the identified gene has been cloned.

In whole of the above process, the selection of T11M primer will amplify the poly A tail region of mRNA. Poly A tail is always attached to 3′ end of the gene and hence T11M primer in combination with an arbitrary primer would always yield 3′ region of the gene.

### EXAMPLE 6

Checking the Size of the PCR Product:

Once the gene has been cloned and the E. coli has been transformed, it becomes imperative to check if the plasmid has received right size of the PCR product. This can be accomplished by performing colony PCR wherein the colony is lysed and the lysate is subsequently used to perform PCR using the appropriate primers. Amplified product is then analysed on an agarose gel.

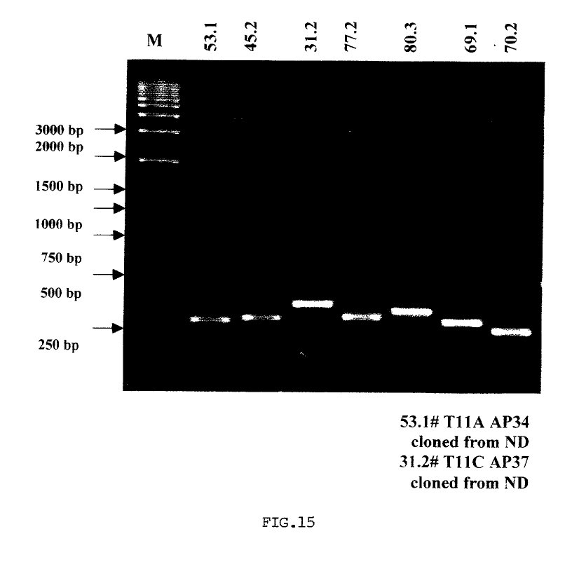

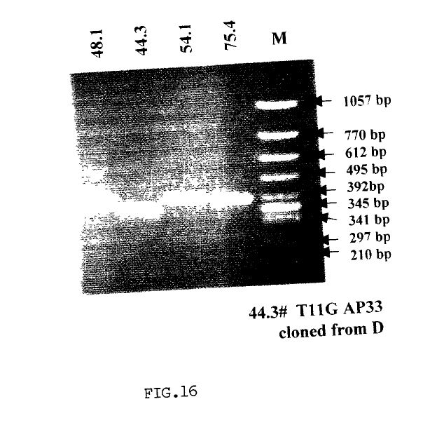

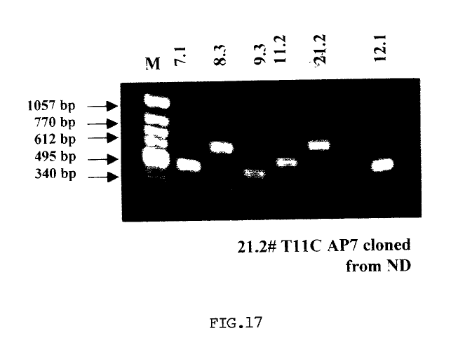

Single isolated colonies were picked up from re-streaked plates (Example 5) and lysed in 50 μl colony lysis buffer (colony lysis buffer: TE (Tris-Cl 10 mM, 1 mM EDTA, pH 8.0) with 0.1% tween 20) by boiling for 10 minutes. Cell debris were pelleted and the supernatant or the colony lysate containing the template DNA was used for PCR. PCR components were essentially the same as in example 4 except that in place of T₁₁M and arbitrary primers Lgh (5′-CGACAACACCGATAATC-3′) and Rgh (5′-GACGCGAACGAAGCAAC-3′) primers (specific to the vector sequences flanking the cloning site) were used and 2 μl of the colony lysate was used in place of eluted DNA. Also the reaction volume was reduced to 20 μl. PCR conditions used for colony PCR were, 94° C. for 30 seconds, →52° C. for 40 seconds, →72° C. for 1 minute for 30 cycles followed by 1 cycle of 5 min extension at 72° C. and final soaking into 4° C. Amplified product are run on 1.5% agarose gel along with molecular weight marker and analyzed for correct size of insert. While using Lgh and Rgh flanking primers, the size of the cloned PCR product was larger by 120 bp due to the flanking vector sequence being amplified (See FIGS. 15-17).

### EXAMPLE 7

Confirmation of the Differential Expression by Northern Blotting

PCR products cloned above represent 3′ end of the differentially expressed genes. Within the scope of the present invention, these cloned fragments of DNA will be called as genes. Since differential display invariably leads to false positives i.e. apparently differentially expressed genes (Wan, J. S. and Erlander, M. G. 1997. Cloning differentially expressed genes by using differential display and subtractive hybridization. In Methods in Molecular Biology. Vol. 85: Differential display methods and protocols. Eds. Liang, P. and Pardee, A. B. Humana press Inc., Totowa, N.J., pp 45-68), a confirmatory test through northern analysis is mandatory to ascertain differential expression between ND and D apical buds. Northern analysis requires preparation of a radio-labelled probe followed by its hybridization with denatured RNA blotted onto a membrane.

Amplified products as in Example 6 were used as a probe in northern analysis After visualising the amplified products on 1.5% agarose gel these were cut from the gel and the DNA was eluted from the gel using QIAEX II gel extraction kit from M/s. Qiagen, Germany following the manufacturer's instructions.

Purified fragments were radiolabelleled with α[³²P]dATP (4000 Ci/mmole) using HotPrime Kit from M/s. GenHunter Corporation, Nashville, USA following their instructions. Radio-labelled probe was purified using QIAquick nucleotide Removal Kit (QIAGEN, Germany) to remove unincorporated radionucleotide.

For blotting, 20 μg of RNA was run on 1.0% formaldehyde agarose gel essentially as described in Example 1. Once the run was completed, gel was washed twice in DEPC trteated autoclaved water for 20 minutes each with shaking. Gel was then washed twice in 10×SSPE (1.5 M sodium chloride, 115 mM NaH₂PO₄, 10 mM EDTA) for 20 minutes each with shaking. In the mean time nylone membrane (boehringer mannheim cat. no.# 1209272) was wetted in DEPC water and then soaked in 10×SSPE (1.5 M sodium chloride, 115 mM NaH₂PO₄, 10 mM EDTA) for 5 minutes with gentle shaking. RNA from the gel was then vacuum-blotted (using pressure of 40 mbar) onto nylon membrane using DEPC-treated 10×SSPE (1.5 M sodium chloride, 115 mM NaH₂PO₄, 10 mM EDTA) as a transfer medium. Transfer was carried out for 4 hours. Pressure was Increased to 70 mbar for 15 minutes before letting out the gel from the vacuum blotter.

After the transfer, gel was removed, and the location of RNA marker was marked on the nylon surface under a UV light source. Membrane was dried and baked at 80° C. for 45 minutes. After a brief rinse in 5×SSPE (20×SSPE: 3M sodium chloride, 230 mM sodium phosphate, 20 mM EDTA) membrane was dipped into prehybridization solution (50% formamide, 0.75 M NaCl, 50 mM sodium phosphate, pH 7.4, 5 mM EDTA, 0.1% Ficoll-400, 0.1% BSA, 0.1% polyvinypyrollidone, 0.1% SDS solution and 150 ug/ml freshly boiled salmon sperm DNA) for 5 hours.

Radiolabelled probe synthesized earlier was denatured by boiling for 10 minutes followed by addition to the prehybridization solution dipping the blotted membrane. Hybridization was carried out for 16 hours. Solution was removed and the membrane was washed twice with 1×SSC (20×SSC; 3M sodium chloride and 0.3M sodium citrate dihydrate, pH, 7.0) containing 0.1% SDS at room temperature for 15 minutes each. Final washing was done at 50° C. using pre-warmed 0.25×SSC containing 0.1% SDS for 15 minutes. Membrane was removed, wrapped in saran wrap and exposed to X-ray film for 12-240 hours depending upon the intensity of the signal.

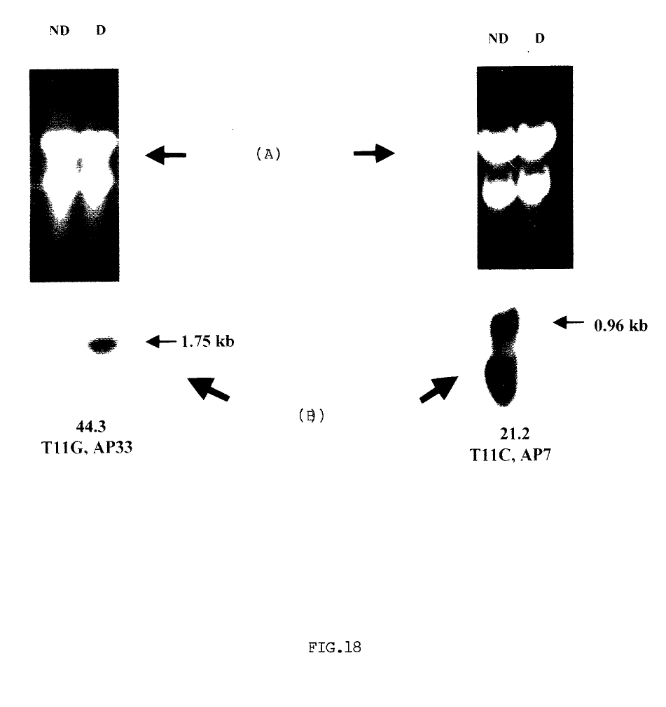

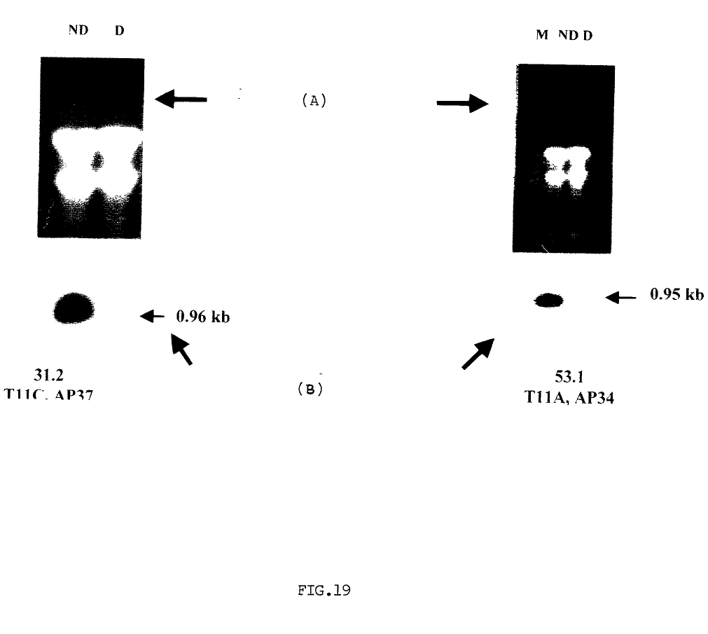

While performing northern hybridization, RNA from ND and D apical buds are blotted on the membrane and tested for the probe of choice. FIGS. 18-19 show the results with 4 such probe and confirm differential expression between ND and D apical buds. 31.2 (T11C, AP37) which is basically a 3′ end region of the gene, hybridized to the transcripts 0.96 kb size on northern blot as in FIG. 19.

21.2 (T11C, AP7) which is basically a 3′ end region of the gene, hybridized to the transcripts 0.96 kb size on northern blot as in FIG. 18.

53.1 (T11A, AP34) which is basically a 3′ end region of the gene, hybridized to the transcripts 0.95 kb size on northern blot as in FIG. 19.

44.3 (T11G, AP33) which is basically a 3′ end region of the gene, hybridized to the transcripts 1.75 kb size on northern blot as in FIG. 18.

### EXAMPLE 8

Method to Correlate the Identified Gene with the Dormancy of Tea Buds:

Above Example 6 identified 4 differentially expressed genes cloned by us. To further correlate these differentially expressed genes with the phenomenon of dormancy, the dormant bush during winter month was forced to break bud dormancy using a plant growth regulator gibberrellic acid (hereinafter referred to GA₃). GA₃ was dissolved in 5% ethanol to yield a final concetration of 5 μM. The solution was applied onto each dormant bud with the help of a paint brush at least 4 times on the same day during the month of December. Bud length and its perimeter was recorded regularly as an indicator of its growth. Out of 20 buds only 10 buds showed increase in growth (dormancy break) in response to GA₃ application. These were termed as forced ND apical buds. These were collected and stored as mentioned in Example 1 and used for the purpose of RNA isolation to be used in northern analysis.

For such an experiment, RNA from ND, D and forced ND was blotted onto a nylon membrane and probed with ND (31.2) probe probe. Various procedures involved are already mentioned in Example 6.

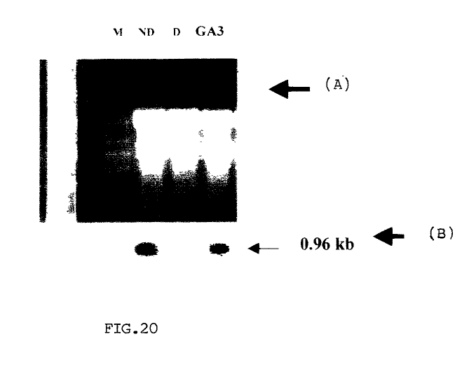

As can be seen from FIG. 20 that the expression of the gene for the probe was down-regulated (i.e. showed lesser expression) in D apical buds compared to the ND. Again the gene showed up-regulation (i.e. showed over-expression) in the forced ND. Thus, it proved that the gene 31.2 (sequence ID No. 1, in the present invention as detailed in Example 8) is indeed a gene related to dormancy. Similar approach can be adopted for other genes as well.

### EXAMPLE 9

Sequencing the Identified Clones:

Each clone was sequenced manually using a T7 sequenase version 2 sequencing kit from M's. Amersham Pharmacia Biotech, USA. Sequencing primers used were [Lgh (5′-CGACAACACCGATAATC-3′) or Rgh (5′-GACGCGAACGAAGCAAC-3′)]

```
(1) INFORMATION FOR SEQ ID NO: 1
    (i) SEQUENCE CHARACTERISTICS:
        (A) LENGTH: 305base pairs
        (B) TYPE: nucleic acid
        (C) STRANDEDNESS: double
        (D) TOPOLOGY: circular
    (ii) MOLECULE TYPE: cDNA
    (iii) SEQUENCE DESCRIPTION: SEQ ID NO: 1
Seq ID No. 1 # Sequence of 31.2 clone:
5′-ATCGCCGTAA TTGCCATGTT TTCCCTCTCA CCGGAATCCT ACG
TTATCC CCTTACCTTC GTGAACATTA CAGTAGGAAT CGGTGGTCCA
ATTATCAACT TAATTTTGGG CGCATCTGTT CGTGTTAACT
AGAAGCCATG TATACATACA ATACAACATG GTTCACTCCT
CCTACAGATT ATGAGTTGAA CTTTTATAAT AAGTTGTAAT
AATGGCTTCT GAATAAGGAG AAGAGGAGCC TCTGTTTGTT
TTACTTATTA CAGATGTGAT ATCGTTCAAC AACTTTGATT
CTGCGAAAAA AAAAA-3′
(2) INFORMATION FOR SEQ ID NO:2
    (i) SEQUENCE CHARACTERISTICS:
        (A) LENGTH: 297 base pairs
        (B) TYPE: nucleic acid
        (C) STRANDEDNESS: double
        (D) TOPOLOGY: circular
    (ii) MOLECULE TYPE: cDNA
    (iii) SEQUENCE DESCRIPTION: SEQ ID NO: 2
Seq ID No. 2 # Sequence of 21.2 Clone:
5′-AGAAGTACCT GAAAGGAAGC TTAACGAGGT GAACATCCAT
TGCAGCCAGC CCTGGAATCT GTACAGGGCA ACTCTGAACC
GGAATTATTT TAATAACCCG TGGGCAATGA TTGCAATTAT
GGCTCGTTTG GTATTACTTC TACTCACTTA GACACAACTG
TATTTACGGT TTTCGCTGGA ATTGTAATTG TTGGAGCGAC
AAAATAGATG GTCACAACTT ATTGGTGAGA GTATCAGTGT
GCTCTTCTTT ATCGTCTTTA ACTCTCCGTG GTAATTACTT
TGACAATATT CATACAT-3′
(3) INFORMATION FOR SEQ ID NO: 3
    (i) SEQUENCE CHARACTERISTICS:
        (A) LENGTH: 239 base pairs
        (B) TYPE: nucleic acid
        (C) STRANDEDNESS: double
        (D) TOPOLOGY: circular
    (ii) MOLECULE TYPE: cDNA
    (iii) SEQUENCE DESCRIPTION: SEQ ID NO: 3
Sequence ID No. 3 # 53.1 Clone:
5′-GAGACTCAGC TCAGCAATCA TGTTCTAAGT GAATGTCACT
CTATCGCCTT CTTGTCCCTC TTAGACATAC TACATCCTCA
TTCTGCTAGA AATGAACTCA TGTAGGTTTT GAAGTTGGGA
ACTTTTGAAA CTGTGTTGTT TGGGTGCTGT CTGTTATACA
ATTCTCTCAA CTGCGGAGAA TTGACGTTGG TTGTAGTGGA
ATTCAACACT TGGGTTTTGT TCTTAGTTAA AAAAAAAAA-3′
(4) INFORMATION FOR SEQ ID NO: 4
    (i) SEQUENCE CHARACTERISTICS:
        (A) LENGTH: 228 base pairs
        (B) TYPE: nucleic acid
        (C) STRANDEDNESS: double
        (D) TOPOLOGY: circular
      (ii) MOLECULE TYPE: cDNA
      (iii) SEQUENCE DESCRIPTION: SEQ ID NO: 4
Seq ID No. 4 # Sequence of 44.3 Clone:
5′-ATAGCTTAGT CACGTGTCTC TTGAGAATGG ACTACGTAGT
TGTTAAGTTG GGTGATCAGA AGGCGTTGAT GATGAATGTA
TGAAGCAGAG ACTACTGAAT GTAATTTTGT TGTTGAAAGA
TGAATGATTT ATTAATGCCT GCATATCTTT CTATTGTTTG
ATGCCAAACC TTTGGGCACA TTTTTTCTTT CTTTTTGTGA
TAATGTTCTC TTCTTGCAAA AAAAAAAA-3′
```

### EXAMPLE 10

Analysis of the Sequences:

Each clone was subjected to BLAST analysis and the clones were found to be unique.

The Main Advantage of the Present Invention are:

(a) Novel DNA sequences expressed and repressed during winter dormancy in the apical buds of Camellia sinensis L. (O.) Kuntze (tea) bush under field conditions have been cloned

(b) These sequences have been cloned from the same genetic make up of the tea bush

(c) A spectrum of 3′ ends of the expressed and repressed genes in non-dormant and dormant apical buds of tea bush growing under field conditions has been presented

(d) Confirmation of the identified 3′ ends of the differentially expressed gene(s) for establishing differential expression during winter dormancy in the apical buds of tea bush growing under field conditions has been carried out.

(e) Method to correlate the identified gene with the dormancy of tea buds has been described under field conditions

(f) Sequencing of the cloned 3′ ends of the differentially expressed gene(s) showed uniqueness in terms of novel sequences not deposited in the data bank so far

We claim:

1. A Novel DNA sequences expressed and repressed during winter dormancy in the apical buds of Camellia sinensis L. (O.) Kuntze (tea) bush or a tree species, said sequences comprising sequence ID 1 to 4 as shown here below:
SEQ ID NO: 1
5′-ATCGCCGTAA TTGCCATGTT TTCCCTCTCA CCGGAATCCT ACG
TTATCC CCTTACCTTC GTGAACATTA CAGTAGGAAT CGGTGGTCCA
ATTATCAACT TAATTTTGGG CGCATCTGTT CGTGTTAACT
AGAAGCCATG TATACATACA ATACAACATG GTTCACTCCT
CCTACAGATT ATGAGTTGAA CTTTTATAAT AAGTTGTAAT
AATGGCTTCT GAATAAGGAG AAGAGGAGCC TCTGTTTGTT
TTACTTATTA CAGATGTGAT ATCGTTCAAC AACTTTGATT
CTGCGAAAAA AAAAA-3′
SEQ ID NO: 2
5′-AGAAGTACCT GAAAGGAAGC TTAACGAGGT GAACATCCAT
TGCAGCCAGC CCTGGAATCT GTACAGGGCA ACTCTGAACC
GGAATTATTT TAATAACCCG TGGGCAATGA TTGCAATTAT
GGCTCGTTTG GTATTACTTC TACTCACTTA GACACAACTG
TATTTACGGT TTTCGCTGGA ATTGTAATTG TTGGAGCGAC
AAAATAGATG GTCACAACTT ATTGGTGAGA GTATCAGTGT
GCTCTTCTTT ATCGTCTTTA ACTCTCCGTG GTAATTACTT
TGACAATATT CATACAT-3′
SEQ ID NO: 3
5′-GAGACTCAGC TCAGCAATCA TGTTCTAAGT GAATGTCACT
CTATCGCCTT CTTGTCCCTC TTAGACATAC TACATCCTCA
TTCTGCTAGA AATGAACTCA TGTAGGTTTT GAAGTTGGGA
ACTTTTGAAA CTGTGTTGTT TGGGTGCTGT CTGTTATACA
ATTCTCTCAA CTGCGGAGAA TTGACGTTGG TTGTAGTGGA
ATTCAACACT TGGGTTTTGT TCTTAGTTAA AAAAAAAAA-3′
SEQ ID NO: 4
5′-ATAGCTTAGT CACGTGTCTC TTGAGAATGG ACTACGTAGT
TGTTAAGTTG GGTGATCAGA AGGCGTTGAT GATGAATGTA
TGAAGCAGAG ACTACTGAAT GTAATTTTGT TGTTGAAAGA
TGAATGATTT ATTAATGCCT GCATATCTTT CTATTGTTTG
ATGCCAAACC TTTGGGCACA TTTTTTCTTT CTTTTTGTGA
TAATGTTCTC TTCTTGCAAA AAAAAAAA-3′

2. The Novel DNA sequences as claimed in claim 1 which are cloned from the tea bush of the same genetic make up.

3. The Novel DNA sequences as claimed in claim 1 which are cloned from the tea bush of the same genetic make up growing under field conditions.

4. Novel DNA sequences as claimed in claim 1, are associated with winter dormancy in tea.

5. Method to clone Novel DNA sequences as claimed in claim 1 from the tree species tea of the same genetic make up growing under field conditions.

6. Novel DNA sequences as claimed in claim 1, which are cloned by any methods but not limited to, subtractive hybridization and differential screening.

7. Novel DNA sequences as claimed in claim 1 wherein, the nucleotide sequence of the DNA is given in SEQ ID NO: 1.

8. Novel DNA sequences as claimed in claim 7 with the nucleotide sequence of the DNA as is given in SEQ ID NO: 1 is overexpressed only in non-dormant apical buds of tea.

9. Novel DNA sequences as claimed in claim 1 wherein, the nucleotide sequence of the DNA is given in SEQ ID NO:2

10. Novel DNA sequences as claimed in claim 9 with the nucleotide sequence of the DNA as is given in SEQ ID NO: 2 is expressed only in non-dormant apical buds of tea.

11. Novel DNA sequences as claimed in claim 1 wherein the nucleotide sequence of the DNA is given in SEQ ID NO: 3

12. Novel DNA sequences as claimed in claim 11 with the nucleotide sequence of the DNA as is given in SEQ ID NO:3 is expressed only in non-dormant apical buds of tea.

13. Novel DNA sequences as claimed in claim 1 wherein, the nucleotide sequence of the DNA is given in SEQ ID NO: 4

14. Novel DNA sequences as claimed in claim 13 with the nucleotide sequence of the DNA as is given in SEQ ID NO: 4 is expressed only in dormant apical buds of tea.

15. Novel sequences as claimed in claim 1 which are capable of being cloned to full-length cDNA.

16. Novel sequences as claimed in claim 1, which are capable of being cloned to full length genomic DNA.

17. Novel sequences as claimed in claim 1, which are capable of being cloned to important sequences, such as but not limited to, promoter sequences and regulatory sequences etc.,

18. Use of sequence data as claimed in claim 1, important information on the gene regulation can be obtained.

19. Use of genes as claimed in claim 1, wherein it is possible to modulate winter dormancy in plants after transferring these genes using the techniques such as, but not limited to, Agrobacterium mediated transformation and Biallistic medited transformation

20. Use of genes as claimed in claim 1, wherein it is possible to modulate winter dormancy in the plants such as, but not limited to, tea, plums, cherries, peaches, Taxus, apples, peers, vines, grapes, olives, Kiwi fruit, figs, morus, strawberries, raspberries, cranberies, blackberries, loganberries, almonds, walnuts and chestnuts after transferring these genes using the techniques such as, but not limited to, Agrobacterium mediated transformation and bialistic medited transformation.

21. Use of sequence data as claimed in claim 1, wherein important information on the gene regulation can be obtained to be exploited to regulate gene expression in transgene.

22. Use of cDNAs and the genomic DNAs as claimed in claims 15-16, for synthesizing unique proteins.

23. Use of unique proteins as claimed in claim 22 for raising antibodies.

24. Use of antibodies as claimed in claim 23, as probe to look for the similar proteins in other plants, animal and/or microbial systems or the like.

25. Use of novel sequences as claimed in claim 1 and cDNAs and the genomic DNAs as claimed in claims 15-16, as probe to look for the sequences of nucleotides in other plants, animal and/or microbial systems and the like.

26. Use of novel sequences as claimed in claim 1 and cDNAs and the genomic DNAs as claimed in claims 15-16, as probe to look for the expression of these sequences of nucleotides in other plants, animal and/or microbial systems and the like.

27. A method to correlate the identified gene with the process of dormancy of tea buds as described for sequence ID 1 is unique.

28. A method as claimed in claim 27 which can be applied to other sequence ID as well.

29. A method as claimed in claim 27 which can be applied to other crops such as, but not limited to plums, cherries, peaches, Taxus, apples, peers, vines, grapes, olives, Kiwi fruit, figs, morus, strawberries, raspberries, cranberries, blackberries, loganberries, almonds, walnuts and chestnuts as well for correlating similar genes.


SEQUENCE LISTING (33 sequences)

SEQ ID NO: 1 - DNA, 304 bp, Camellia sinensis L. (O.) Kuntze
atcgccgtaattgccatgttttccctctcaccggaatcctacgttatccccttaccttcgtgaacattacagtaggaatcggtggtccaattatcaacttaattttgggcgcatctgttcgtgttaactagaagccatgtatacatacaatacaacatggttcactcctcctacagattatgagttgaacttttataataagttgtaataatggcttctgaataaggagaagaggagcctctgtttgttttacttattacagatgtgatatcgttcaacaactttgattctgcgaaaaaaaaaa

SEQ ID NO: 2 - DNA, 297 bp, Camellia sinensis L. (O.) Kuntze
agaagtacctgaaaggaagcttaacgaggtgaacatccattgcagccagccctggaatctgtacagggcaactctgaaccggaattattttaataacccgtgggcaatgattgcaattatggctcgtttggtattacttctactcacttagacacaactgtatttacggttttcgctggaattgtaattgttggagcgacaaaatagatggtcacaacttattggtgagagtatcagtgtgctcttctttatcgtctttaactctccgtggtaattactttgacaatattcatacat

SEQ ID NO: 3 - DNA, 239 bp, Camellia sinensis L. (O.) Kuntze
gagactcagctcagcaatcatgttctaagtgaatgtcactctatcgccttcttgtccctcttagacatactacatcctcattctgctagaaatgaactcatgtaggttttgaagttgggaacttttgaaactgtgttgtttgggtgctgtctgttatacaattctctcaactgcggagaattgacgttggttgtagtggaattcaacacttgggttttgttcttagttaaaaaaaaaaa

SEQ ID NO: 4 - DNA, 228 bp, Camellia sinensis L. (O.) Kuntze
atagcttagtcacgtgtctcttgagaatggactacgtagttgttaagttgggtgatcagaaggcgttgatgatgaatgtatgaagcagagactactgaatgtaattttgttgttgaaagatgaatgatttattaatgcctgcatatctttctattgtttgatgccaaacctttgggcacattttttctttctttttgtgataatgttctcttcttgcaaaaaaaaaaa

SEQ ID NO: 5 - DNA, 18 bp, Artificial Sequence
  primer_bind
aagcttttttttttttta

SEQ ID NO: 6 - DNA, 18 bp, Artificial Sequence
  primer_bind
aagctttttttttttttc

SEQ ID NO: 7 - DNA, 18 bp, Artificial Sequence
  primer_bind
aagctttttttttttttg

SEQ ID NO: 8 - DNA, 13 bp, Artificial Sequence
  primer_bind
aagcttgattgcc

SEQ ID NO: 9 - DNA, 13 bp, Artificial Sequence
  primer_bind
aagcttcgactgt

SEQ ID NO: 10 - DNA, 13 bp, Artificial Sequence
  primer_bind
aagctttggtcag

SEQ ID NO: 11 - DNA, 13 bp, Artificial Sequence
  primer_bind
aagcttctcaacg

SEQ ID NO: 12 - DNA, 13 bp, Artificial Sequence
  primer_bind
aagcttagtaggc

SEQ ID NO: 13 - DNA, 13 bp, Artificial Sequence
  primer_bind
aagcttgcaccat

SEQ ID NO: 14 - DNA, 13 bp, Artificial Sequence
  primer_bind
aagcttaacgagg

SEQ ID NO: 15 - DNA, 13 bp, Artificial Sequence
  primer_bind
aagcttttaccgc

SEQ ID NO: 16 - DNA, 13 bp, Artificial Sequence
  primer_bind
aagcttgctgctc

SEQ ID NO: 17 - DNA, 13 bp, Artificial Sequence
  primer_bind
aagcttcagcagc

SEQ ID NO: 18 - DNA, 13 bp, Artificial Sequence
  primer_bind
aagcttcagggca

SEQ ID NO: 19 - DNA, 13 bp, Artificial Sequence
  primer_bind
aagcttcgacgct

SEQ ID NO: 20 - DNA, 13 bp, Artificial Sequence
  primer_bind
aagcttgggccta

SEQ ID NO: 21 - DNA, 13 bp, Artificial Sequence
  primer_bind
aagcttccagtgc

SEQ ID NO: 22 - DNA, 13 bp, Artificial Sequence
  primer_bind
aagctttcccagc

SEQ ID NO: 23 - DNA, 13 bp, Artificial Sequence
  primer_bind
aagcttgtcagcc

SEQ ID NO: 24 - DNA, 13 bp, Artificial Sequence
  primer_bind
aagcttcaagacc

SEQ ID NO: 25 - DNA, 13 bp, Artificial Sequence
  primer_bind
aagcttgccttta

SEQ ID NO: 26 - DNA, 13 bp, Artificial Sequence
  primer_bind
aagctttatttat

SEQ ID NO: 27 - DNA, 13 bp, Artificial Sequence
  primer_bind
aagcttctttggt

SEQ ID NO: 28 - DNA, 13 bp, Artificial Sequence
  primer_bind
aagcttaataacg

SEQ ID NO: 29 - DNA, 13 bp, Artificial Sequence
  primer_bind
aagctttcatatg

SEQ ID NO: 30 - DNA, 13 bp, Artificial Sequence
  primer_bind
aagcttgtagtaa

SEQ ID NO: 31 - DNA, 13 bp, Artificial Sequence
  primer_bind
aagctttcaaaga

SEQ ID NO: 32 - DNA, 17 bp, Artificial Sequence
  primer_bind
cgacaacaccgataatc

SEQ ID NO: 33 - DNA, 17 bp, Artificial Sequence
  primer_bind
gacgcgaacgaagcaac